(19) United States
(12) Patent Application Publication
(10) Pub. No.: US 2010/0298714 A1
(43) Pub. Date: Nov. 25, 2010

(54) ULTRASONIC DIAGNOSTIC APPARATUS AND ULTRASONIC PROBE

(75) Inventor: Yasuo Miyajima, Utsunomiya-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/781,275

(22) Filed: May 17, 2010

(30) Foreign Application Priority Data
May 19, 2009 (JP) 2009-121209
Apr. 8, 2010 (JP) 2010-089231

(51) Int. Cl. A61B 8/14

(52) U.S. Cl. 600/459

(57) ABSTRACT

When ultrasonic pulses are transmitted to the inside of an examined subject by driving transducer elements of which the quantity is Mx and that are arranged in a two-dimensional array within an ultrasonic probe, a signal selecting unit included in the probe selects, for each of the transducer elements, two adjacent driving signals each having a time delay closest to an accurate time delay (an ideal time delay) required to drive the transducer element, out of driving signals in as many channels as Mo (Mo<<Mx) supplied from a transmitting and receiving unit in an apparatus main body while time delays thereof are quantized by Δτ. Subsequently, a weighting unit weights each of the selected adjacent driving signals by employing a variable capacity device. A synthesizing and separating unit generates a synthesized driving signal having the ideal time delay, by performing an additive synthesis process on the weighted adjacent driving signals.

### CROSS-REFERENCE TO RELATED APPLICATIONS

This application is based upon and claims the benefit of priority from the prior Japanese Patent Application No. 2009-121209, filed on May 19, 2009, and Japanese Patent Application No. 2010-089231, filed on Apr. 8, 2010; the entire contents of both of which are incorporated herein by reference.

### BACKGROUND

1. Field

Embodiments described herein related generally to an ultrasonic diagnostic apparatus and an ultrasonic probe.

2. Description of the Related Art

Ultrasonic diagnostic apparatuses are configured so as to emit ultrasonic pulses generated from transducer elements provided in an ultrasonic probe into the inside of an examined subject and to collect biological information by receiving, through the transducer elements, ultrasonic reflected waves caused by differences in acoustic impedances of the tissues of the examined subject. Further, because ultrasonic diagnostic apparatuses are capable of displaying ultrasonic image data in a real-time manner through a simple operation of bringing the ultrasonic probe into contact with the examined subject, ultrasonic diagnostic apparatuses are widely used in morphological diagnoses and functional diagnoses of various types of organs.

A type of ultrasonic diagnostic apparatus that is most popularly used these days is configured in such a manner that a plurality of transducer elements are arranged in a one-dimensional array within an ultrasonic probe, so that two-dimensional image data is displayed in a real-time manner by exercising high-speed control on time delays of driving signals to be supplied to the transducer elements or of reception signals obtained from the transducer elements. In addition, another type of ultrasonic diagnostic apparatus that has recently been developed uses an ultrasonic probe in which the transducer elements are arranged in a two-dimensional array, so that it is possible to display, in a real-time manner, image data in a three-dimensional space or on an arbitrary sliced cross-sectional plane of an examined subject.

As a method for collecting image data by using transducer elements that are arranged in a two-dimensional array, a method is known by which an ultrasonic scanning process is performed in an arbitrary direction within a three-dimensional space, by controlling the time delays of the driving signals to be supplied to the transducer elements and of the reception signals obtained from the transducer elements. In addition, as a method for collecting image data by using transducer elements that are arranged in a two-dimensional array, another method is also known by which an ultrasonic scanning process is performed in a predetermined direction (i.e., an X direction) by controlling the time delays in the manner described above, whereas an ultrasonic scanning process is performed in a direction (i.e., a Y direction) perpendicular to the predetermined direction while electronically moving an aperture (i.e., moving a transducer element group that is used during the ultrasonic transmitting/receiving process).

According to any of these methods, however, the number of transducer elements significantly increases (i.e., 10 to 100 times) when the transducer elements are arranged in a two-dimensional array. As a result, in the case where an electronic circuit is not built into the ultrasonic probe, both the number of channels of signal cables provided between the ultrasonic probe and the apparatus main body as well as the number of channels in the transmission circuit and the reception circuit provided in the apparatus main body significantly increase, along with the increase in the number of transducer elements. For this reason, it has been considered extremely difficult to realize an apparatus that is excellent in operability and has, for example, a circuit scale, a size, a weight, and a price that are practical.

To cope with the problem described above, methods for having an electronic circuit built into an ultrasonic probe have been researched. For example, a method has been proposed by which a plurality of transducer elements that are arranged in a two-dimensional array are divided into sections of a predetermined size so as to form a plurality of sub-arrays, so that sub-arrays that are positioned at a substantially equal distance from a focus point during a transmitting process or a receiving process are brought into a common connection by using a switch provided within an ultrasonic probe. By using this method, it is possible to reduce the number of channels of the signal cables and the number of channels in the transmission circuit and the reception circuit provided in the apparatus main body.

According to the method described above, however, it is necessary to install an extremely large number of switches and the like in the limited space within the ultrasonic probe. Thus, a stringent limitation is imposed on the size of an area for installation of a switching circuit and the electric power consumption (heat generation). As a result, it becomes difficult to supply a sufficient level of driving voltage to the transducer elements, and that can be a cause of a degradation of a Signal/Noise (S/N) ratio of the image data.

Further, the resolution of the time delay of the driving signals supplied to the transducer elements and the resolution of the time delay used in a phase-matching and adding process performed on the reception signals obtained from the transducer elements depend on the number of channels of the signal cables. Accordingly, in the case where the number of channels is reduced by bringing the transducer elements into a common connection according to the method described above, a transmission directivity property of the ultrasonic pulse and a reception directivity property of the ultrasonic reflected wave are degraded. In other words, the method described above has a problem where it is not possible to obtain image data having excellent spatial resolution performance and/or excellent contrast resolution performance.

### BRIEF DESCRIPTION OF THE DRAWINGS

FIG. 1 is a block diagram of an entirety of an ultrasonic diagnostic apparatus according to an embodiment;

FIG. 2 is a drawing for explaining a structure of an ultrasonic probe according to the embodiment;

FIG. 3 is a drawing for explaining specific examples of transducer elements to which mutually the same adjacent driving signals are supplied according to the embodiment;

FIG. 4 is a drawing for explaining a structure of each of variable capacity devices (Micro Electro Mechanical Systems [MEMS]) that are used in a weighting unit according to the embodiment;

FIG. 5 is a chart for explaining capacitance characteristics of the variable capacity devices that are used in the weighting unit according to the embodiment;

FIG. 6 is a diagram for explaining a signal selecting unit, the weighting unit, a synthesizing and separating unit, and a driving circuit that are used during a transmitting process according to the embodiment;

FIG. 7 is a block diagram of a specific configuration of a transmitting and receiving unit included in an ultrasonic diagnostic apparatus according to the embodiment;

FIG. 8 is a diagram of a specific circuit configuration of a phase-matching and adding unit included in a receiving unit according to the embodiment;

FIG. 9 is a block diagram of a specific configuration of an ultrasonic data generating unit included in the ultrasonic diagnostic apparatus according to the embodiment;

FIG. 10 is a diagram for explaining specific circuit constants used by the weighting unit included in the ultrasonic probe according to the embodiment;

FIG. 11 is a chart for explaining specific examples of adjacent driving signals supplied to the ultrasonic probe according to the embodiment and synthesized driving signals generated based on the adjacent driving signals;

FIG. 12 is a diagram for explaining specific circuit constants used by the weighting unit during a receiving process according to the embodiment;

FIG. 13 is a diagram of specific examples of the signal selecting unit, the weighting unit, and the synthesizing and separating unit that are suitable for performing a transmitting process according to the embodiment;

FIG. 14 is a table for explaining ideal time delays of synthesized driving signals to be supplied to the transducer elements according to the embodiment;

FIG. 15 is a drawing for explaining a process to divide a transducer element group into sections when a circuit configuration of the signal selecting unit is determined according to the embodiment;

FIG. 16 is a diagram of a specific circuit configuration of the signal selecting unit according to the embodiment;

FIG. 17 is a diagram for explaining an adjacent driving signal selecting process performed by the signal selecting unit according to the embodiment; and

FIG. 18 is a block diagram of an entirety of an ultrasonic diagnostic apparatus according to a modification example of the embodiment.

### DETAILED DESCRIPTION

In one embodiment, an ultrasonic diagnostic apparatus includes an ultrasonic probe, a transmitting unit, a receiving unit, and an image data generating unit. The ultrasonic probe includes a plurality of transducer elements that are arranged in an array and each of which is configured so as to transmit an ultrasonic pulse into an ultrasonic transmission/reception direction that is set for an examined subject and to convert an ultrasonic reflected wave arriving from the ultrasonic transmission/reception direction into a reception signal. The transmitting unit supplies driving signals that are in a plurality of channels and have discrete time delays to the ultrasonic probe. The receiving unit performs a phase-matching and adding process on second reception signals that are in a plurality of channels, have discrete time delays, and have been generated based on first reception signals obtained from the transducer elements. The image data generating unit that generates image data based on ultrasonic data obtained by processing reception signals resulting from the phase-matching and adding process. And, the ultrasonic probe includes a signal selecting unit, a weighting unit, and a synthesizing and separating unit. The signal selecting unit selects, as adjacent driving signals, driving signals that are in at least two channels and each of which has a time delay close to an ideal time delay required to drive each of the transducer elements, out of the driving signals that are in the plurality of channels and are supplied from the transmitting unit. The weighting unit applies a predetermined weight to each of the selected adjacent driving signals. The synthesizing and separating unit generates a synthesized driving signal having the ideal time delay by synthesizing the adjacent driving signals to which the predetermined weights have been applied and drives each of the transducer elements.

In the following sections, exemplary embodiments will be explained, with reference to the accompanying drawings.

According to the exemplary embodiments described below, an ultrasonic pulse is transmitted to the inside of the body of an examined subject (hereinafter, “subject”) by driving transducer elements of which the quantity is equal to Mx and that are arranged in a two-dimensional array within an ultrasonic probe. According to the exemplary embodiments, during an ultrasonic pulse transmitting process, for each of the transducer elements, driving signals that are in two channels (hereinafter, “adjacent driving signals”) and each of which has a time delay closest to a time delay (hereinafter, an “ideal time delay”) required to drive the transducer element are selected, out of driving signals that are in as many channels as Mo (where Mo<<Mx) and are supplied from a transmitting and receiving unit included in an apparatus main body while the time delays thereof are quantized by Δτ. After that, according to the exemplary embodiments, a driving signal (hereinafter, a “synthesized driving signal”) having the ideal time delay is generated by performing a weighting process on each of the selected adjacent driving signals while employing variable capacity devices and subsequently performing an additive synthesis process on the weighted adjacent driving signals, so that it is possible to drive the transducer elements by using the obtained synthesized driving signals.

In the description of the exemplary embodiments below, examples will be explained in which B-mode image data and color Doppler image data that are two dimensional on an arbitrary sliced cross-sectional plane are generated by using an ultrasonic probe in which a plurality of transducer elements are arranged in a two-dimensional array; however, the exemplary embodiments may be applied to examples in which other types of two-dimensional image data are generated. Further, the exemplary embodiments may be applied to examples in which three-dimensional image data, Multi-planar Reconstruction (MPR) image data, Maximum Intensity Projection (MIP) image data, or the like is generated based on volume data obtained by performing a three-dimensional scanning process while using the ultrasonic probe described above.

### <A Configuration of the Apparatus>

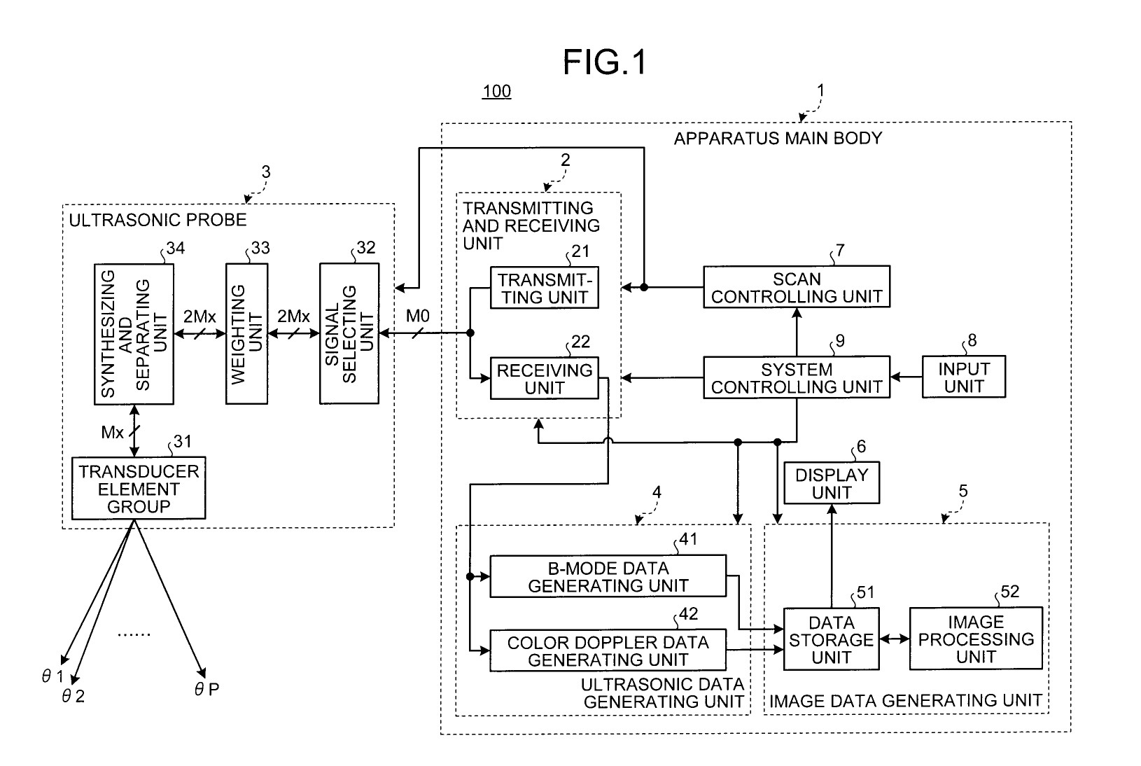

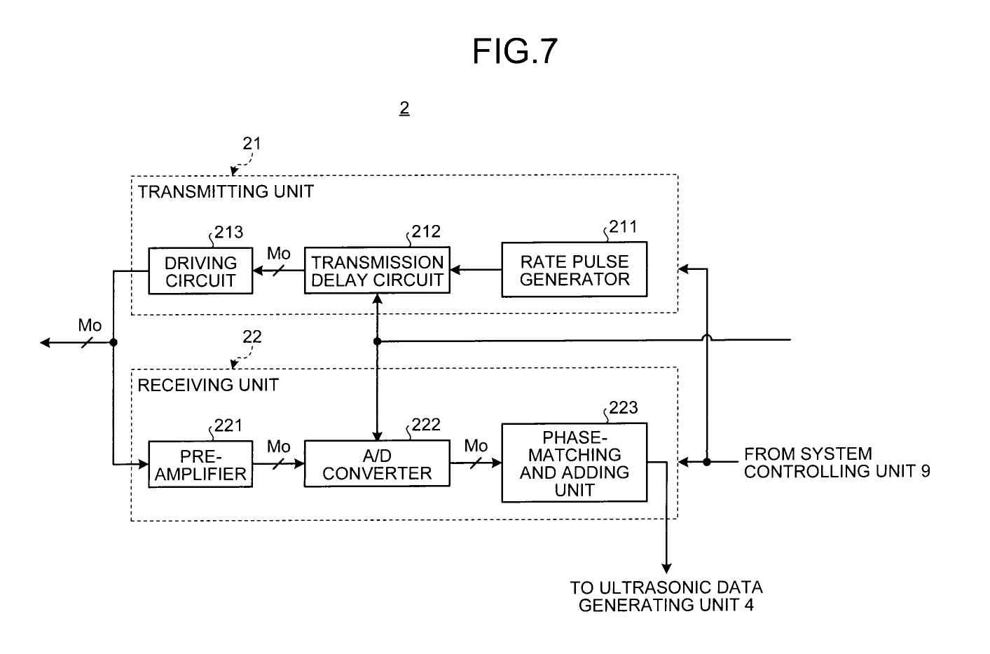

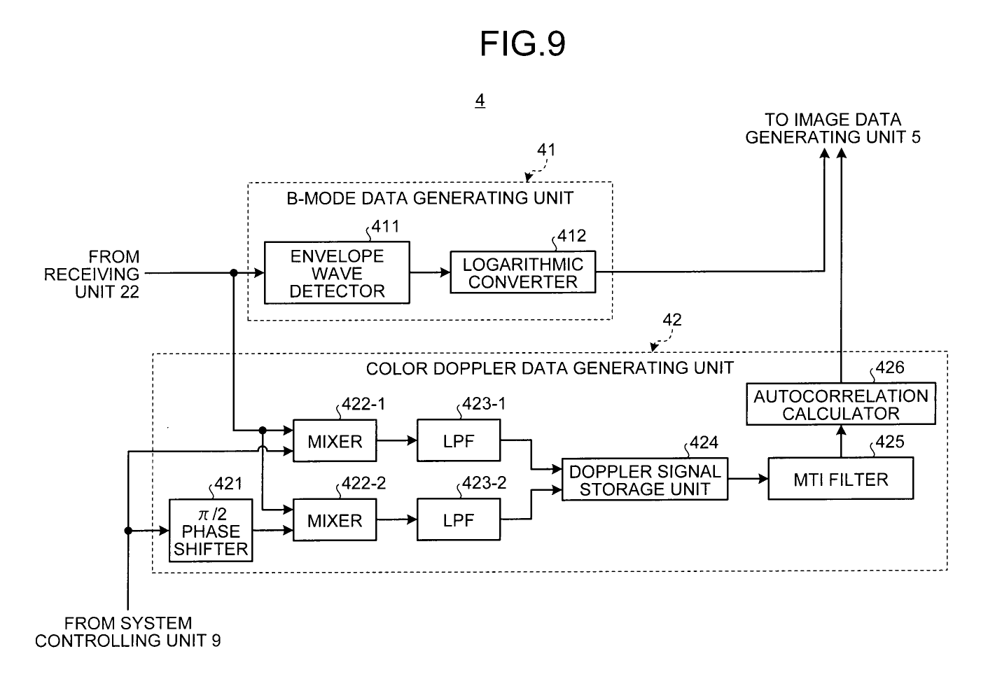

A configuration of an ultrasonic diagnostic apparatus according to an embodiment of the present invention and operations of functional units will be explained, with reference to FIGS. 1 to 17. FIG. 1 is a block diagram of an entirety of the ultrasonic diagnostic apparatus. FIGS. 7 and 9 are block diagrams of specific configurations of a transmitting and receiving unit and an ultrasonic data generating unit that are included in the ultrasonic diagnostic apparatus.

An ultrasonic diagnostic apparatus 100 shown in FIG. 1 includes an ultrasonic probe 3, a transmitting and receiving unit 2, an ultrasonic data generating unit 4, an image data generating unit 5, and a display unit 6. The ultrasonic probe 3 includes a plurality of transducer elements of which the quantity is equal to Mx (hereinafter, a “transducer element group”) and that are arranged in a two-dimensional array. The ultrasonic probe 3 performs an ultrasonic transmitting/receiving process on a subject. The transmitting and receiving unit 2 supplies driving signals (i.e., first driving signals) that are in as many channels as Mo (where Mo<<Mx) and of which time delays are quantized by Δτ, to the ultrasonic probe 3. Also, the transmitting and receiving unit 2 applies time delays quantized by Δτ to reception signals that are in as many channels as Mo and have been obtained from the ultrasonic probe 3 and performs a phase-matching and adding process thereon. The ultrasonic data generating unit 4 generates B-mode data and color Doppler data as ultrasonic data, by performing a signal processing process on the reception signals on which the phase-matching and adding process has been performed and that have been supplied from the transmitting and receiving unit 2. The image data generating unit 5 generates B-mode image data and color Doppler image data that are two dimensional, by sequentially storing the ultrasonic data that has been generated by the ultrasonic data generating unit 4, while keeping each piece of ultrasonic data in correspondence with an ultrasonic transmission/reception direction. The display unit 6 displays the B-mode image data and the color Doppler image data that have been generated by the image data generating unit 5.

Further, the ultrasonic diagnostic apparatus 100 includes a scan controlling unit 7, an input unit 8, and a system controlling unit 9. The scan controlling unit 7 performs, for example, a time delay controlling process for the purpose of performing an ultrasonic transmitting/receiving process with respect to a predetermined direction θp as well as a selection controlling process on the driving signals and the reception signals. The input unit 8 inputs information about the subject, sets conditions for generating ultrasonic data and conditions for generating image data, and inputs various types of command signals and the like. The system controlling unit 9 controls the functional units described above that are included in the ultrasonic diagnostic apparatus 100 in an integrated manner. Further, the ultrasonic probe 3 is connected to the transmitting and receiving unit 2 and the scan controlling unit 7 that are included in an apparatus main body 1 via a multi-core cable (not shown).

The ultrasonic probe 3 includes a transducer element group 31, a signal selecting unit 32, a weighting unit 33, and a synthesizing and separating unit 34. In the description of the exemplary embodiments, the ultrasonic probe 3 is for a sector scanning purpose and includes the transducer elements of which the quantity is equal to Mx and that are arranged in a two-dimensional array; however, an ultrasonic probe that is capable of performing a linear scanning process, a convex scanning process, or the like is also applicable.

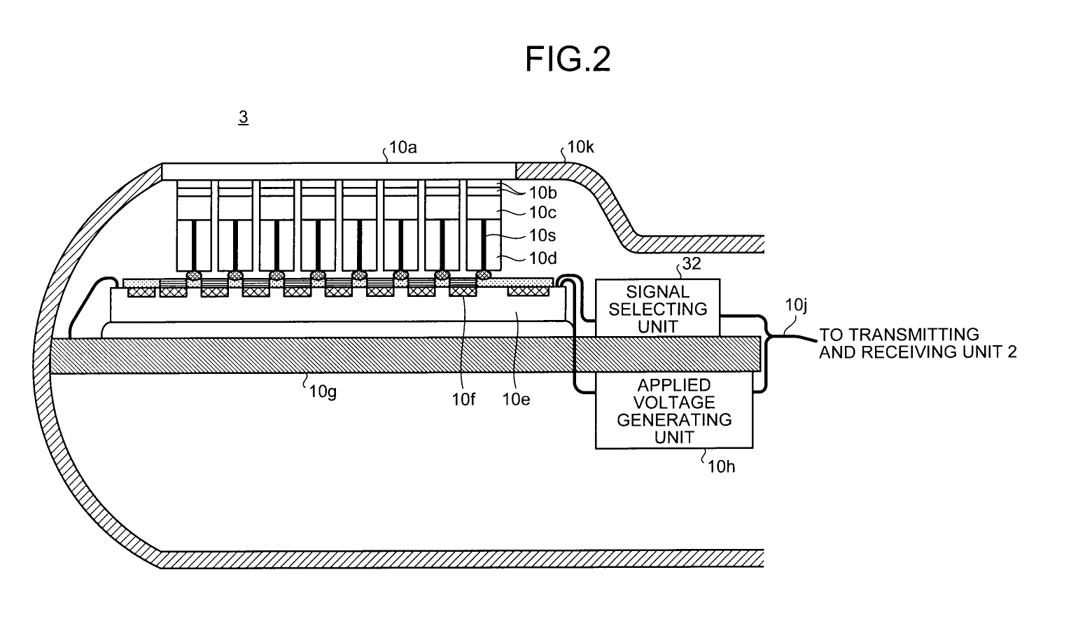

Next, in particular, as a specific example of the ultrasonic probe 3 for which there are demands to make the size smaller and to reduce the number of channels in the multi-core cable, the ultrasonic probe 3 for a transesophageal ultrasonic examination purpose will be explained with reference to FIG. 2. By using the ultrasonic probe 3 for a transesophageal ultrasonic examination purpose that performs an ultrasonic transmitting/receiving process while using the transducer elements inserted in the esophagus of a subject, it is possible to collect good-quality image data of the heart, without being significantly influenced by the costal bones the lung field, and the like.

As shown in FIG. 2, the transducer element group 31 is formed by transducer elements 10c of which the quantity is equal to Mx and that are arranged in a two-dimensional array along an ultrasonic transmission/reception surface of the ultrasonic probe 3. Each of the transducer elements 10c is an electro-acoustic converting element and has a function of converting an electric driving signal into an ultrasonic pulse (i.e., a transmitted ultrasound wave) during a transmitting process and converting an ultrasonic reflected wave into an electric reception signal during a receiving process.

Further, on a front surface (i.e., the ultrasonic transmission/reception surface) on which the transducer elements 10c forming the transducer element group 31 are grounded, two matching layers 10b that are for the purpose of efficiently performing the ultrasonic transmitting/receiving process on the subject by matching acoustic impedances, as well as an acoustic lens 10a for the purpose of causing the ultrasonic pulse and the ultrasonic reflected wave to focus at a predetermined depth are attached. The acoustic lens 10a is fixed to an aperture of a resin cover 10k.

In addition, on a rear surface of the transducer elements 10c, a backing member 10d that absorbs unnecessary ultrasound waves emitted rearwards from the transducer elements 10c and that holds the transducer elements 10c is provided. Each of signal lines 10s that are connected to the rear surfaces of the transducer elements 10c goes through the inside of the backing member 10d and is connected, via a bump connection, to one of terminals of a corresponding one of variable capacity devices (i.e., Micro Electro Mechanical Systems [MEMS]) 10f that are provided on a surface of a silicon substrate 10e by using a micromachine technique.

The other terminal of each of the variable capacity devices 10f is connected to one of terminals of the signal selecting unit 32 provided on a printed wiring board 10g, via a signal line (not shown) provided on the silicon substrate 10e. The other terminal of the signal selecting unit 32 is connected to the transmitting and receiving unit 2 provided in the apparatus main body 1 via a multi-core cable 10j.

Further, on the printed wiring board 10g included in the ultrasonic probe 3, an applied voltage generating unit 10h that supplies a predetermined direct-current voltage to a position between electrodes (explained later) included in the variable capacity devices is provided. A voltage control signal for the applied voltage generating unit 10h and a selection control signal for the signal selecting unit 32 are supplied from the scan controlling unit 7 included in the apparatus main body 1 shown in FIG. 1, via the multi-core cable 10j.

The synthesizing and separating unit 34 included in the ultrasonic probe 3 shown in FIG. 1 is configured by, for example, connecting transducer-element-side terminals of the variable capacity devices 10f in parallel. Further, the weighting unit 33 included in the ultrasonic probe 3 shown in FIG. 1 is configured with the variable capacity devices 10f as well as the applied voltage generating unit 10h that supplies the direct-current voltage to the positions between the electrodes of the variable capacity devices 10f, and the like.

Further, the signal selecting unit 32 has, during a transmitting process, a function of selecting, for each of the transducer elements, adjacent driving signals that are in two channels and each of which has a time delay closest to the ideal time delay, out of driving signals that are in as many channels as Mo and are supplied from the transmitting and receiving unit 2 included in the apparatus main body 1 while the time delays thereof are quantized by Δτ. Furthermore, the signal selecting unit 32 has, during a receiving process, a function of generating reception signals (i.e., second reception signals) that are in as many channels as Mo and of which the time delays are quantized by Δτ, by selecting/synthesizing reception signals (i.e., first reception signals) that are in as many channels as Mx and have been obtained from the transducer elements of which the quantity is equal to Mx.

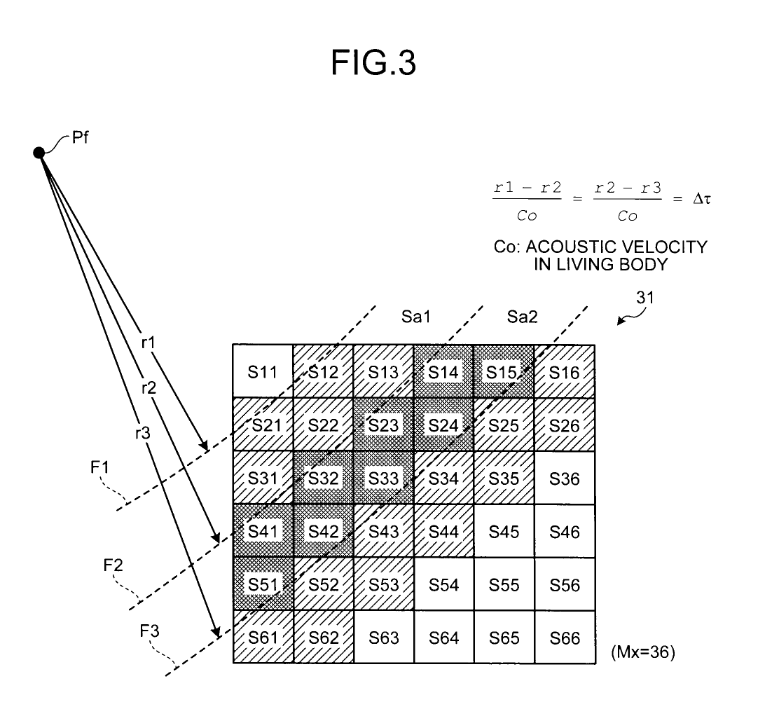

FIG. 3 is a drawing for explaining the transducer elements to which mutually the same adjacent driving signals are supplied. Shown in FIG. 3 are the transducer element group 31 in which the transducer elements of which the quantity is equal to Mx are arranged in a two-dimensional array as well as a point Pf that is a focus point of the ultrasonic pulses emitted into predetermined directions from the transducer element group 31 or a focus point of ultrasonic reflected waves obtained from predetermined directions. In the following sections, to keep the explanation simple, the transducer element group 31 in which the transducer elements (S11 to S66) of which the quantity is Mx=36 are arranged in a two-dimensional array will be explained; however, the present embodiment is not limited to this example. In actuality, the transducer element group 31 is formed by a larger number of transducer elements.

In the present example, the signal selecting unit 32 sets, for instance, a radius r1 and a radius r2 so that a difference between a propagation time of τ1 and a propagation time of τ2 becomes equal to the time delay of Δτ, the propagation time τ1 (while τ1=r1/Co is satisfied, where Co: an acoustic velocity in a living body) being a time period it takes for an ultrasound wave reflected at the focus point Pf to reach a spherical surface F1 having the radius r1; and the propagation time of τ2 (while τ2=r2/Co is satisfied) being a time period it takes for an ultrasound wave reflected at the focus point Pf to reach a spherical surface F2 having the radius r2. Further, the signal selecting unit 32 selects the transducer elements S12, S13, S21, and so on out of the transducer element group 31 that are present in a region Sa1 defined as being positioned between the spherical surface F1 and the spherical surface F2. Subsequently, the signal selecting unit 32 supplies adjacent driving signals that are namely a driving signal St1 having a time delay of τo1 and a driving signal St2 having a time delay of τo2 (where τo2=τo1−Δτ) to each of the selected transducer elements, via the weighting unit 33 and the synthesizing and separating unit 34.

Similarly, the signal selecting unit 32 sets a radius r3 so that a difference between a propagation time of τ3 (while τ3=r3/Co is satisfied) and the propagation time of τ2 becomes equal to the time delay of Δτ, the propagation time of τ3 being a time period it takes for an ultrasound wave reflected at the focus point Pf to reach a spherical surface F3 having the radius r3. Further, the signal selecting unit 32 selects the transducer elements S14, S15, S23, S24 and so on that are present in a region Sa2 defined as being positioned between the spherical surface F2 and the spherical surface F3. Subsequently, the signal selecting unit 32 supplies the driving signal St2 having the time delay of τo2 and a driving signal St3 having a time delay of τo3 (where τo3=τo2−Δτ) to each of the selected transducer elements, via the weighting unit 33 and the synthesizing and separating unit 34.

In the same manner as described above, the signal selecting unit 32 supplies adjacent driving signals that are in two channels and that have a time delay difference Δτ to each of all the transducer elements forming the transducer element group 31, via the weighting unit 33 and the synthesizing and separating unit 34. A specific method for selecting the adjacent driving signals to be supplied to each of the transducer elements will be explained later.

As explained with reference to FIG. 2 above, the weighting unit 33 included in the ultrasonic probe 3 shown in FIG. 1 includes the variable capacity devices 10f and the applied voltage generating unit 10h that supplies the direct-current voltage to the positions between the electrodes of the variable capacity devices 10f. The weighting unit 33 applies a weight to the adjacent driving signals to be supplied to the transducer elements and the reception signals obtained from the transducer elements, based on capacitances of the variable capacity devices 10f that change according to the levels of the voltage applied thereto.

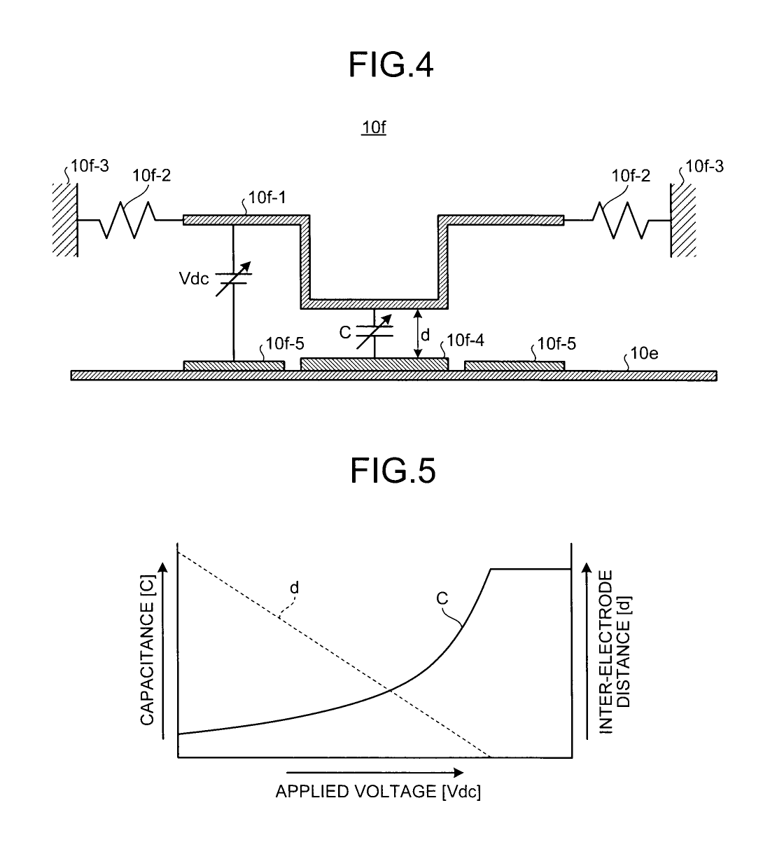

FIG. 4 is a drawing of a specific example of a variable capacity device (MEMS) that has newly been developed by introducing a micromachine technique, which has advanced rapidly in recent years. Two ends of a movable electrode 10f-1 included in a variable capacity device 10f are supported by a mechanical suspension 10f-2 connected to a supporting unit 10f-3. Further, within the variable capacity device 10f, a capacitor is formed between the movable electrode 10f-1 and a fixed electrode 10f-4, by causing a central part (i.e., a projecting part) of the movable electrode 10f-1 to be positioned close to the fixed electrode 10f-4 provided on the silicon substrate 10e.

Further, the variable capacity device 10f is configured so that, when a direct-current voltage Vdc is applied to a position between the movable electrode 10f-1 and a bias electrode 10f-5 provided on the silicon substrate 10e, an electrostatic force is generated between the electrodes. A distance d between the movable electrode 10f-1 and the fixed electrode 10f-4 is updated by the generated electrostatic force. In other words, the variable capacity device 10f is configured so that it is possible to obtain a desired capacitance C (while C=εS/d is satisfied, where ε: an electric permittivity of an inter-electrode medium; S: an electrode area of the central part; and d: an inter-electrode distance) by controlling the direct-current voltage Vdc applied to the position between the movable electrode 10f-1 and the bias electrode 10f-5. In FIG. 5, levels of the capacitance C and the inter-electrode distance d with respect to the applied voltage Vdc are shown.

After the weights based on an impedance determined according to the capacitance of the variable capacity device 10f have been applied to the adjacent driving signals in the two channels that have been selected by the signal selecting unit 32 out of the driving signals in as many channels as Mo, an additive synthesis process is performed thereon by the synthesizing and separating unit 34, and subsequently the result of the additive synthesis process is supplied to corresponding ones of the transducer elements. The synthesizing and separating unit 34 may be configured with an adder circuit. Alternatively, in the case where the impedance of each of the transducer elements is low, it is possible to perform the additive synthesis process on the adjacent driving signals in the two channels that have been weighted, by simply connecting the transducer-element-side terminals in the weighting unit 33 in parallel. According to this method, because the electric power consumed by the variable capacity devices 10f that are used for weighing the adjacent driving signals is extremely small, it is possible to significantly reduce the electric power consumed by the ultrasonic probe 3 and the heat generated by the ultrasonic probe 3, compared to an example in which a weighting circuit is configured with resistors.

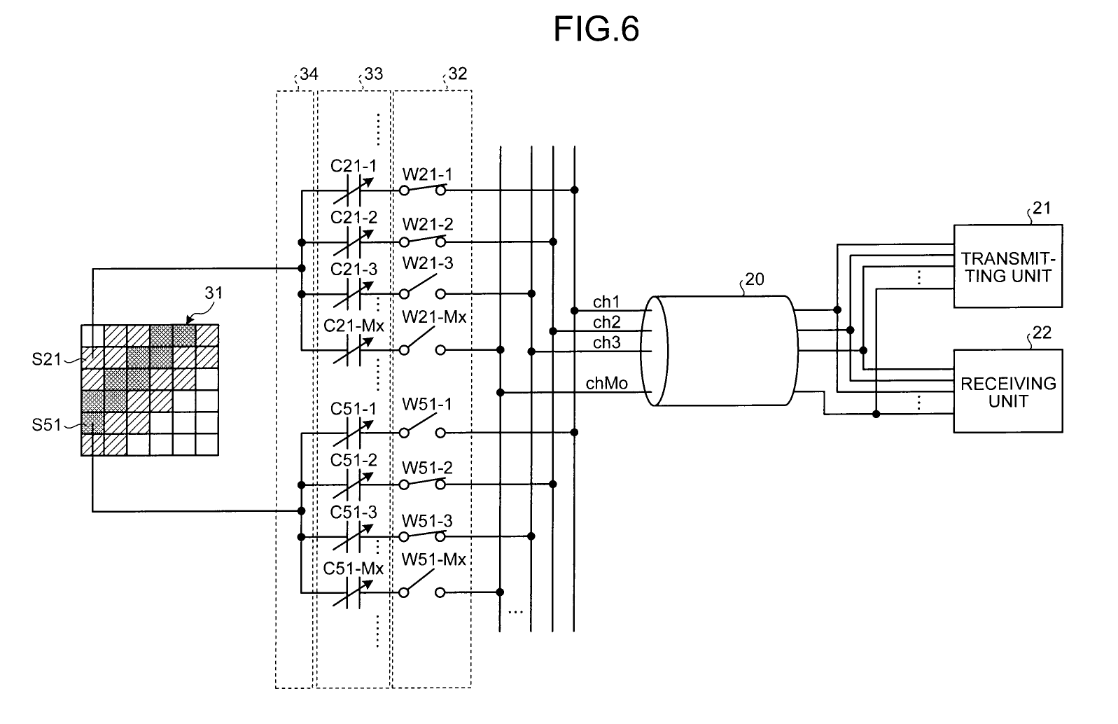

Next, a specific example of the ultrasonic probe 3 including the transducer element group 31, the signal selecting unit 32, the weighting unit 33, and the synthesizing and separating unit 34 will be explained, with reference to FIG. 6. With reference to FIG. 6, an example will be explained in which adjacent driving signals in two channels that have been selected by the signal selecting unit 32 are supplied to each of the transducer elements S21 and S51, the transducer element S21 being present in the region Sa1 defined as being positioned between the spherical surfaces of which the distances from the focus point Pf are r1 and r2, respectively; and the transducer element S51 being present in the region Sa2 defined as being positioned between the spherical surfaces of which the distances from the focus point Pf are r2 and r3, respectively.

In the present example, a transmitting unit 21 included in the apparatus main body 1 supplies the driving signal St1 having the time delay of τo1, via a channel ch1 in a multi-core cable 20. Also, the transmitting unit 21 included in the apparatus main body 1 supplies the driving signal St2 having the time delay of τo2 (where τo2=τo1−Δτ) and the driving signal St3 having the time delay of τo3 (where τo3=τo2−Δτ), via a channel ch2 and a channel ch3, respectively.

Further, based on a selection control signal supplied from the scan controlling unit 7 shown in FIG. 1, the signal selecting unit 32 causes each of switches W21-1 and W21-2 to be in an electrically conductive state. As a result, the signal selecting unit 32 selects the driving signal St1 and the driving signal St2 as the adjacent driving signals out of the driving signals that are in as many channels as Mo and are supplied from the transmitting unit 21, and the signal selecting unit 32 supplies the adjacent driving signals that have been selected to variable capacity devices C21-1 and C21-2 included in the weighting unit 33.

Further, the applied voltage generating unit 10h included in the weighting unit 33 generates a predetermined capacitance by applying a direct-current voltage that has been generated based on a voltage control signal supplied from the scan controlling unit 7, to a position between the electrodes of the variable capacity devices C21-1 and C21-2. More specifically, the applied voltage generating unit 10h included in the weighting unit 33 applies a weight to each of the adjacent driving signals St1 and St2 by using the generated capacitance. Further, the synthesizing and separating unit 34 generates a synthesized driving signal St21 having an ideal time delay by performing an additive synthesis process on the adjacent driving signals St1 and St2 that have been weighted, and the synthesizing and separating unit 34 supplies the generated synthesized driving signal St21 to the transducer element S21.

Similarly, based on a selection control signal supplied from the scan controlling unit 7, the signal selecting unit 32 causes each of switches W51-2 and W51-3 to be in an electrically conductive state. As a result, the signal selecting unit 32 selects the driving signal St2 and the driving signal St3 as the adjacent driving signals out of the driving signals that are in as many channels as Mo and are supplied from the transmitting unit 21, and the signal selecting unit 32 supplies the adjacent driving signals that have been selected to variable capacity devices C51-2 and C51-3 included in the weighting unit 33.

Further, the applied voltage generating unit 10h included in the weighting unit 33 generates a predetermined capacitance by applying a direct-current voltage that has been generated based on a voltage control signal supplied from the scan controlling unit 7, to a position between the electrodes of the variable capacity devices C51-2 and C51-3. More specifically, the applied voltage generating unit 10h included in the weighting unit 33 applies a weight to each of the adjacent driving signals St2 and St3 by using the generated capacitance. Further, the synthesizing and separating unit 34 generates a synthesized driving signal St51 having an ideal time delay by performing an additive synthesis process on the adjacent driving signals St2 and St3 that have been weighted, and the synthesizing and separating unit 34 supplies the generated synthesized driving signal St51 to the transducer element S51.

In the same manner as described above, to each of all the transducer elements forming the transducer element group 31, the adjacent driving signals in two channels having the time delay difference Δτ are weighted, added together, and supplied. In this situation, by controlling the capacitances of the variable capacity devices included in the weighting unit 33, it is also possible to incorporate the switching function of the signal selecting unit 32 at the same time. A specific example of the synthesized driving signals that are newly generated by the weighting unit 33 and the synthesizing and separating unit 34 will be explained later.

In contrast, during a receiving process, each of the reception signals (i.e., the first reception signals) that are in as many channels as Mx and are supplied from the transducer element group 31 is further separated by the synthesizing and separating unit 34 into reception signals in two channels, so that a weighting process that is the same as the one performed during the transmitting process is performed thereon by the weighting unit 33. Subsequently, the signal selecting unit 32 generates the reception signals (i.e., the second reception signals) that are in as many channels as Mo and of which the time delays are quantized by Δτ, by performing a selecting and synthesizing process on the reception signals in as many channels as 2Mx that have been weighted. Subsequently, the obtained reception signals that are in as many channels as Mo are supplied to the transmitting and receiving unit 2 included in the apparatus main body 1 via the multi-core cable 20.

Next, the transmitting and receiving unit 2 shown in FIG. 1 will be explained with reference to FIG. 7. The transmitting and receiving unit 2 includes: the transmitting unit 21 that supplies the driving signals that are in as many channels as Mo and of which the time delays are quantized by Δτ to the ultrasonic probe 3; and a receiving unit 22 that performs the phase-matching and adding process on the second reception signals that are in as many channels as Mo and are supplied from the ultrasonic probe 3. The transmitting unit 21 includes a rate pulse generator 211, a transmission delay circuit 212, and a driving circuit 213. The rate pulse generator 211 generates a rate pulse that determines a repeating cycle of the ultrasonic pulse emitted into the inside of the subject, by dividing a frequency of a reference signal supplied from the system controlling unit 9.

Further, the transmission delay circuit 212 is configured with delay circuits having as many channels as Mo. The transmission delay circuit 212 applies a transmission delay time that is made up of a focus-purpose time delay and a deflection-purpose time delay to the rate pulse and supplies the result to the driving circuit 213. The focus-purpose time delay is used for focusing the ultrasonic pulse at a predetermined depth so as to obtain a narrow beam width during the transmitting process. The deflection-purpose time delay is used for emitting the ultrasonic pulse into the predetermined direction. The time delay of the individual rate pulse that is output from each of as many channels as Mo that are included in the transmission delay circuit 212 is quantized by Δτ. Further, in the case where a maximum time delay that is required to perform an ultrasonic transmitting/receiving process with respect to a maximum deflection angle θP is expressed as τmax, it is possible to determine the number of channels Mo described above by using an expression Mo=τmax/Δτ.

Further, the driving circuit 213 generates the driving signals that are in as many channels as Mo and have predetermined waveforms and predetermined wave amplitude, based on the rate pulse that has the transmission time delay described above and has been supplied from the transmission delay circuit 212. Further, the driving circuit 213 supplies the generated driving signals that are in as many channels as Mo to the signal selecting unit 32 included in the ultrasonic probe 3 via the multi-core cable 20. Each of the driving signals that are generated by the driving circuit 213 may be an impulse wave having a half-wavelength pulse width or may be a pulse wave having a wave train that is equal to or longer than the wavelength. It should be noted, however, that the latter waveform is preferable for the purpose of generating an excellent synthesized driving signal by performing the weighting and the adding processes.

Further, the receiving unit 22 has a function of performing a phase-matching and adding process on the reception signals (i.e., the second reception signals) that are in as many channels as Mo and are supplied from the signal selecting unit 32 included in the ultrasonic probe 3 via the multi-core cable. More specifically, the receiving unit 22 includes a preamplifier 221 having as many channels as Mo, an Analog-Digital (A/D) converter 222, and a phase-matching and adding unit 223. The preamplifier 221 is used for securing a sufficient S/N ratio by amplifying the reception signals that are in as many channels as Mo and are supplied from the signal selecting unit 32. A protecting circuit (not shown) for the purpose of providing protection against a high-voltage driving signal that is output by the driving circuit 213 included in the transmitting unit 21 is provided in an initial stage portion of the preamplifier 221.

Each of the reception signals that have been amplified to a predetermined magnitude by the preamplifier 221 is converted to a digital signal by the A/D converter 222, before being sent to the phase-matching and adding unit 223. By applying a predetermined time delay to the reception signals that are in as many channels as Mo and have been supplied from the A/D converter 222 and performing an additive synthesis process (i.e., the phase-matching and adding process) thereon, the phase-matching and adding unit 223 sets a strong reception directivity onto some of the reception signals from the predetermined direction. It should be noted that the phase-matching and adding unit 223 has a so-called parallel simultaneous receiving function that makes it possible to separate and receive ultrasonic reflected waves that arrive substantially at the same time from a plurality of directions by controlling the time delays with respect to the reception signals in as many channels as Mo.

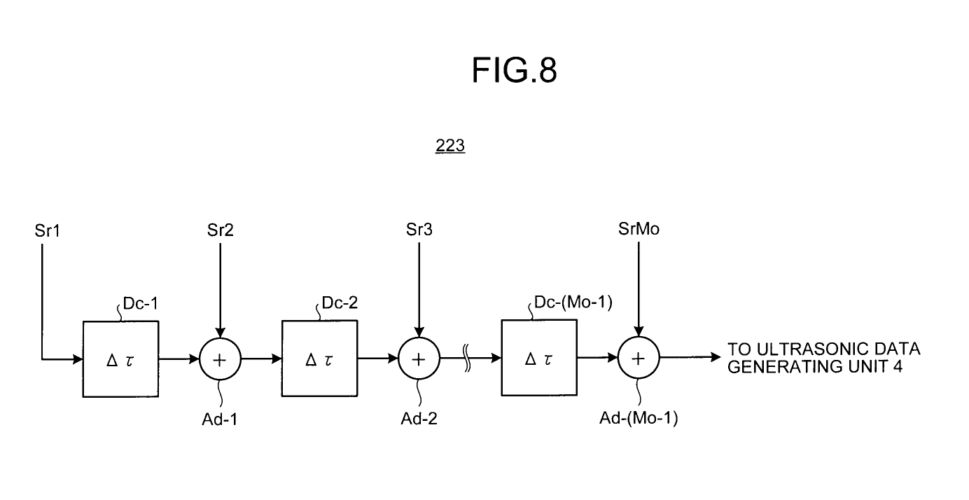

Next, a specific configuration of the phase-matching and adding unit 223 will be explained, with reference to FIG. 8. The phase-matching and adding unit 223 has a configuration that is the same as that of a Finite Impulse Response (FIR) filter, which is a commonly-used digital filter. More specifically, as shown in FIG. 8, the phase-matching and adding unit 223 is configured so that delay circuits Dc-1 to Dc-(Mo-1) of which the total quantity is equal to Mo-1 and each of which applies the time delay of Δτ to an input signal and adder circuits Ad-1 to Ad-(Mo-1) of which the total quantity is equal to Mo-1 are connected in series so as to alternate. Each of reception signals Sr1 to SrMo that are in as many channels as Mo and have been supplied from the A/D converter 222 included in the receiving unit 22 is supplied to a corresponding one of input terminals of the delay circuit Dc-1 and the adder circuits Ad-1 to Ad-(Mo-1) that are included in the phase-matching and adding unit 223.

For example, after the delay circuit Dc-1 applies the time delay of Δτ to the reception signal Sr1 having a reference time delay of τ0, the adder circuit Ad-1 performs an additive synthesis process on the reception signal Sr1 and the reception signal Sr2 having a time delay of τ0+Δτ. After the delay circuit Dc-2 applies the time delay of Δτ to the reception signals Sr1 and Sr2 on which the additive synthesis process has been performed, the adder circuit Ad-2 performs an additive synthesis process on the reception signals Sr1 and Sr2 and the reception signal Sr3 having a time delay of τ0+2Δτ. By performing delaying processes and adding processes on the reception signals Sr1 to SrMo in this manner, it is possible to obtain a result that is the same as a result of applying a time delay of (Mo−m)Δτ to reception signals Srm (where m=1 to Mo) having a time delay of τ0+(m−1)Δτ and performing the additive synthesis process (i.e., the phase-matching and adding process) thereon.

Next, a specific configuration of the ultrasonic data generating unit 4 shown in FIG. 1 will be explained, with reference to a block diagram shown in FIG. 9. The ultrasonic data generating unit 4 includes a B-mode data generating unit 41 and a color Doppler data generating unit 42. The B-mode data generating unit 41 generates the B-mode data by processing B-mode reception signals that are output from the phase-matching and adding unit 223 included in the receiving unit 22. The color Doppler data generating unit 42 detects a Doppler signal by performing a quadrature phase detecting process on color-Doppler-mode reception signals and generates the color Doppler data based on the obtained Doppler signal.

The B-mode data generating unit 41 includes an envelope wave detector 411 and a logarithmic converter 412. The envelope wave detector 411 performs an envelope wave detecting process on the reception signals on which the phase-matching and adding process has been performed and that have been supplied from the phase-matching and adding unit 223 included in the receiving unit 22. The logarithmic converter 412 generates the B-mode data by performing a logarithmic converting process on the amplitudes of the reception signals on which the envelope wave detecting process has been performed.

Further, the color Doppler data generating unit 42 includes a π/2 phase shifter 421, mixers 422-1 and 422-2, and Low Pass Filters (LPFs) 423-1 and 423-2. The π/2 phase shifter 421, the mixers 422-1 and 422-2, and the LPFs 423-1 and 423-2 perform a quadrature phase detecting process on the reception signals on which the phase-matching and adding process has been performed and that have been supplied from the phase-matching and adding unit 223 included in the receiving unit 22 and detect a complex-type Doppler signal that is made up of a real part and an imaginary part.

Further, the color Doppler data generating unit 42 includes a Doppler signal storage unit 424, a Moving Target Indicator (MTI) filter 425, and an autocorrelation calculator 426. The Doppler signals that are output from the LPFs 423-1 and 423-2 as a result of performing the ultrasonic transmitting/receiving process in the mutually same direction a plurality of times are temporarily stored into the Doppler signal storage unit 424. Subsequently, the MTI filter 425, which is a high-pass digital filter, reads Doppler signals in a temporal sequence that have been collected from mutually the same site of the subject out of the Doppler signal storage unit 424 and eliminates, from the read Doppler signals, components (i.e., clutter components) caused by respiratory displacements, pulsatile displacements, and the like of the organs. Further, the autocorrelation calculator 426 performs an autocorrelation calculating process on the Doppler signals corresponding to only blood flow components that have been extracted by the MTI filter 425. As a result, the autocorrelation calculator 426 generates, as the color Doppler data, an average flow rate value of the blood flow, and a velocity distribution value indicating disturbances in the blood flow speed as well as a power value indicating an energy level of the Doppler signals.

Further, the image data generating unit 5 shown in FIG. 1 includes a data storage unit 51 and an image processing unit 52. The data storage unit 51 sequentially stores therein the B-mode data and the color Doppler data that have been generated by the ultrasonic data generating unit 4 in units of ultrasonic transmission/reception directions. The image processing unit 52 generates the B-mode image data and the color Doppler image data that are two-dimensional from the B-mode data and the color Doppler data that have been stored in the data storage unit 51. After that, the image processing unit 52 stores the B-mode image data and the color Doppler image data that have been generated into the data storage unit 51. Further, the image processing unit 52 performs, as necessary, image processing processes such as a filtering process on the B-mode image data and the color Doppler image data that have been stored in the data storage unit 51.

The display unit 6 includes a display data generating unit, a data converting unit, and a monitor (not shown). The display data generating unit generates display data by performing a synthesizing process, a scan converting process, or the like on the B-mode image data and the color Doppler image data that have been generated by the image data generating unit 5. Further, the data converting unit performs a converting process such as a Digital/Analog (D/A) converting process, a television format converting process, or the like on the display data that has been supplied from the display data generating unit and displays the data on the monitor.

The scan controlling unit 7 performs a time delay controlling process on the transmission delay circuit 212 included in the transmitting unit 21 and the phase-matching and adding unit 223 included in the receiving unit 22, for the purpose of transmitting/receiving an ultrasound wave with respect to each of the ultrasonic transmission/reception directions θ1 to θP that have been set for the subject. Further, the scan controlling unit 7 performs, for example, the following controlling processes on the signal selecting unit 32 and the weighting unit 33 that are included in the ultrasonic probe 3: the selection controlling process for the purpose of selecting the adjacent driving signals to be supplied to each of the transducer elements out of the driving signals in as many channels as Mo; the selection controlling process for the purpose of selecting/synthesizing the reception signals that are in as many channels as 2Mx and have been supplied from the weighting unit 33 and generating the reception signals that are in as many channels as Mo and of which the time delays are quantized by Δτ; and the voltage controlling process for the purpose of setting the direct-current voltage to be supplied to the variable capacity devices.

The input unit 8 is configured so as to include input devices provided on an operation panel, such as a display panel, a keyboard, a trackball, a mouse, and selection buttons. For example, the input unit 8 inputs information about the subject, sets conditions for generating and displaying various types of image data, and inputs various types of command signals, by using the display panel and the input devices described above.

The system controlling unit 9 includes a Central Processing Unit (CPU) and a memory circuit (not shown). The information described above that is input/set by a user through the input unit 8 is stored into the memory circuit. Further, the CPU controls the functional units included in the ultrasonic diagnostic apparatus 100 and the entire system in an integrated manner, based on the information described above that has been stored into the memory circuit, so that the B-mode image data and the color Doppler image data on a sliced cross-sectional plane that has arbitrarily been set for the subject are generated and displayed.

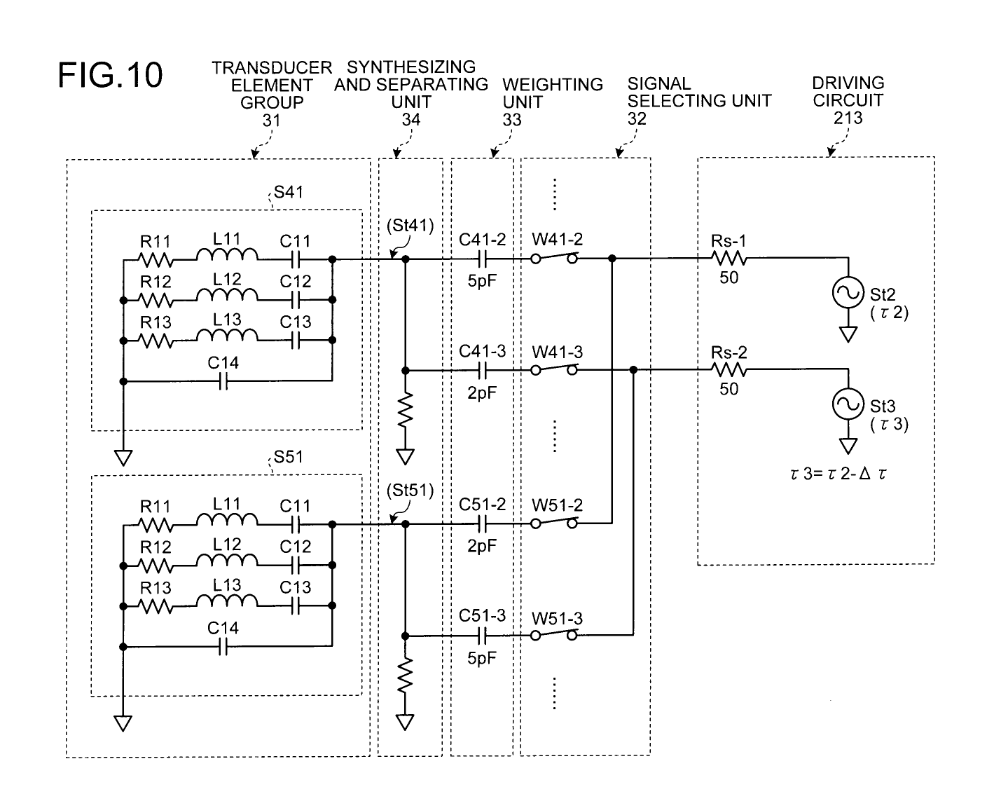

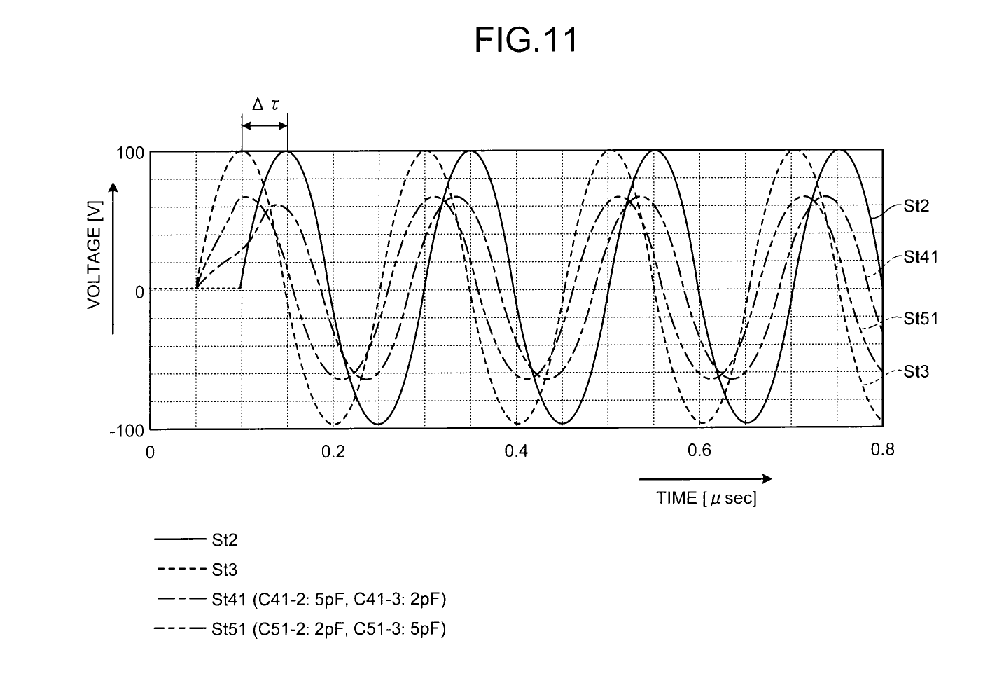

Next, specific examples of the synthesized driving signals that are generated by the weighting unit 33 and the synthesizing and separating unit 34 will be explained, with reference to FIGS. 10 and 11. FIG. 10 is a diagram of specific examples of the transducer element group 31, the signal selecting unit 32, the weighting unit 33, and the synthesizing and separating unit 34 that are included in the ultrasonic probe 3 as well as the driving circuit 213 provided in the transmitting and receiving unit 2 included in the apparatus main body 1. In FIG. 10, to keep the explanation simple, only an example is shown in which the driving circuit 213 included in the transmitting unit 21 supplies the adjacent driving signals St2 and St3 having the time delay difference Δτ to the transducer element S41 and the transducer element S51 (see FIG. 3) that are present in the region Sa2 defined as being positioned between the spherical surface F2 at the distance r2 from the focus point Pf and the spherical surface F3 at the distance r3 from the focus point Pf. The circuit configurations used to depict the transducer element group 31 are equivalent circuits of the transducer circuits that are commonly used for a transducer element analysis or the like.

In the present example, the synthesizing and separating unit 34 generates a synthesized driving signal St41 by performing an additive synthesis process on the adjacent driving signals St2 and St3 that have been weighted by using capacitances of variable capacity devices C41-2 and C41-3 included in the weighting unit 33, and the synthesizing and separating unit 34 supplies the generated synthesized driving signal St41 to the transducer element S41. Similarly, the synthesizing and separating unit 34 generates a synthesized driving signal St51 by performing an additive synthesis process on the adjacent driving signals St2 and St3 that have been weighted by using capacitances of variable capacity devices C51-2 and C51-3 included in the weighting unit 33, and the synthesizing and separating unit 34 supplies the generated synthesized driving signal St51 to the transducer element S51.

Shown in FIG. 11 are specific examples of the adjacent driving signals St41 and St51 that have been generated based on the adjacent driving signals St1 and St2. In the example shown in FIG. 11, for instance, the center frequency fo of the ultrasonic pulse is assumed to be 5 megahertz, while an output impedance of the driving circuit 213 is assumed to be 50 ohms, whereas the time delay difference (i.e., a quantized time delay) Δτ between the adjacent driving signals St2 and St3 is assumed to be To/4=50 nanoseconds (where the cycle To is the reciprocal of the center frequency fo). In FIG. 11, the synthesized driving signals St41 and St51 that are generated based on the adjacent driving signals St1 and St2 are shown in correspondence with a situation where the voltage applied to each of the variable capacity devices is controlled in such a manner that the capacitance of each of the variable capacity devices C41-2 and C51-3 is 5 picoFarads, whereas the capacitance of each of the variable capacity devices C41-3 and C51-2 is 2 picoFarads.

More specifically, by controlling the capacitances of the variable capacity devices, it is possible to configure the time delay of the synthesized driving signal St41 supplied to the transducer element S41 and the time delay of the synthesized driving signal St51 supplied to the transducer element S51 so as to be an arbitrary value between the time delays of the driving signal St2 and the driving signal St3 that are output by the driving circuit 213. Accordingly, the ultrasonic probe 3 is able to supply the synthesized driving signals each having the ideal time delay to the transducer element S41 and the transducer element S51.

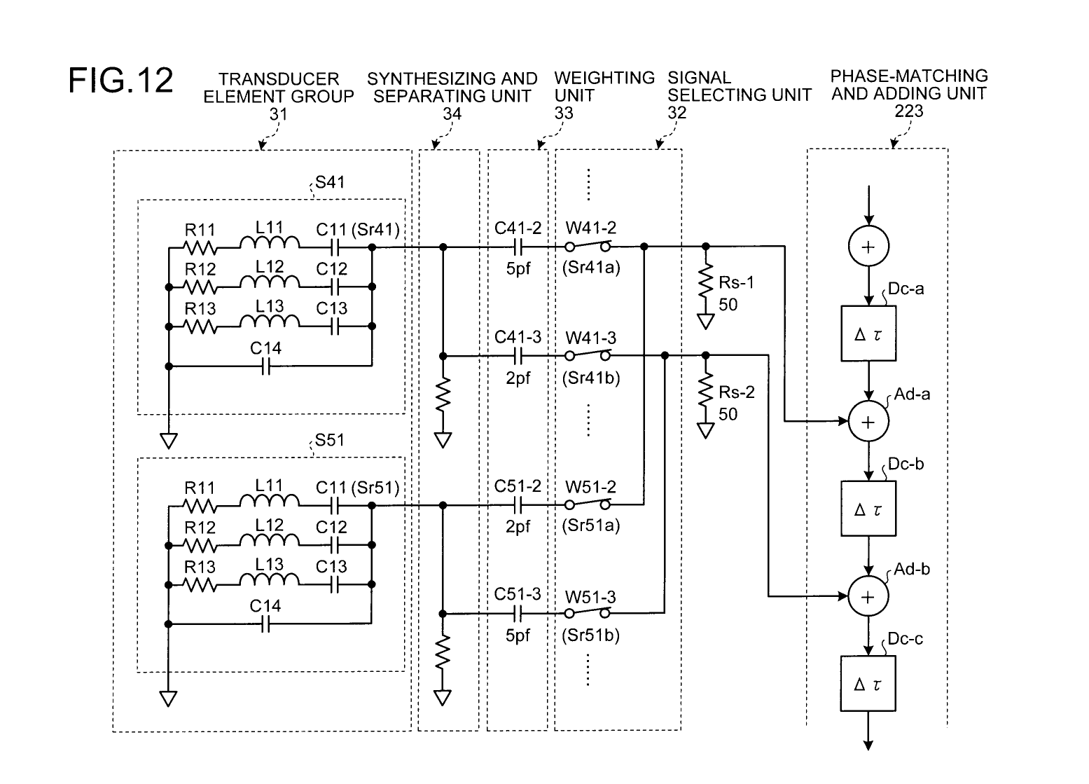

Next, a specific example of the phase-matching and adding process performed on the reception signals that have been weighted by the weighting unit 33 will be explained, with reference to FIG. 12. FIG. 12 is a diagram of specific examples of the transducer element group 31, the signal selecting unit 32, the weighting unit 33, and the synthesizing and separating unit 34 that are included in the ultrasonic probe 3 as well as the phase-matching and adding unit 223 provided in the receiving unit 22 included in the apparatus main body 1. The preamplifier 221 and the A/D converter 222 that are provided at the stages preceding the phase-matching and adding unit 223 are omitted from FIG. 12. In addition, like in FIG. 10, only an example is shown in which the phase-matching and adding unit 223 performs a phase-matching and adding process on the reception signals Sr41 and Sr51 that are obtained from the transducer element S41 and the transducer elements S51 included in the transducer element group 31.

More specifically, the reception signal Sr41 detected by the transducer element S41 is separated into two reception signals Sr41a and Sr41b by the synthesizing and separating unit 34. Similarly, the reception signal Sr51 detected by the transducer element S51 is separated into reception signals Sr51a and Sr51b by the synthesizing and separating unit 34. Further, an additive synthesis process is performed on the reception signal Sr41a that has been weighted by the variable capacity device C41-2 included in the weighting unit 33 and the reception signal Sr51a that has been weighted by the variable capacity device C51-2 included in the weighting unit 33, and the result of the additive synthesis process is supplied to an adder circuit Ad-a included in the phase-matching and adding unit 223. Further, an additive synthesis process is performed on the reception signal Sr41b that has been weighted by the variable capacity device C41-3 included in the weighting unit 33 and the reception signal Sr51b that has been weighted by the variable capacity device C51-3 included in the weighting unit 33, and the result of the additive synthesis process is supplied to an adder circuit Ad-b included in the phase-matching and adding unit 223.

Subsequently, the adder circuit Ad-b synthesizes the reception signals Sr41b and Sr51b that have been weighted with the reception signals Sr41a and Sr51a that have been weighted and that have been delayed by a time equal to Δτ by a delay circuit Dc-b. As a result, the adder circuit Ad-b has performed a phase-matching and adding process on the reception signal Sr41 having the same time delay as the synthesized driving signal St41 shown in FIG. 11 and the reception signal Sr51 having the same time delay as the synthesized driving signal St51 shown in FIG. 11. Similarly, a delay circuit Dc-b and an adder circuit Ad-b perform phase-matching and adding processes on the reception signals obtained from the transducer elements S32, S42, S23, S33, and so on that are present in the region Sa2 that is defined as being positioned between the spherical surface F2 and the spherical surface F2 shown in FIG. 3.

Further, a delay circuit Dc-a and the adder circuit Ad-a perform phase-matching and adding processes on the reception signals obtained from the transducer elements S21, S31, S12, S22, and so on that are present in the region Sa1 that is defined as being positioned between the spherical surface F1 and the spherical surface F2. In addition, the delay circuit Dc-a and the adder circuit Ad-a perform phase-matching and adding processes on the results of the phase-matching and adding process described above and the reception signals obtained from the transducer elements positioned in the region Sa2. In other words, by supplying the reception signals that have been weighted to the predetermined adder circuits, the signal selecting unit 32 is able to accurately perform the phase-matching and adding processes on the reception signals that are in as many channels as Mx and have been obtained from the transducer element group 31.

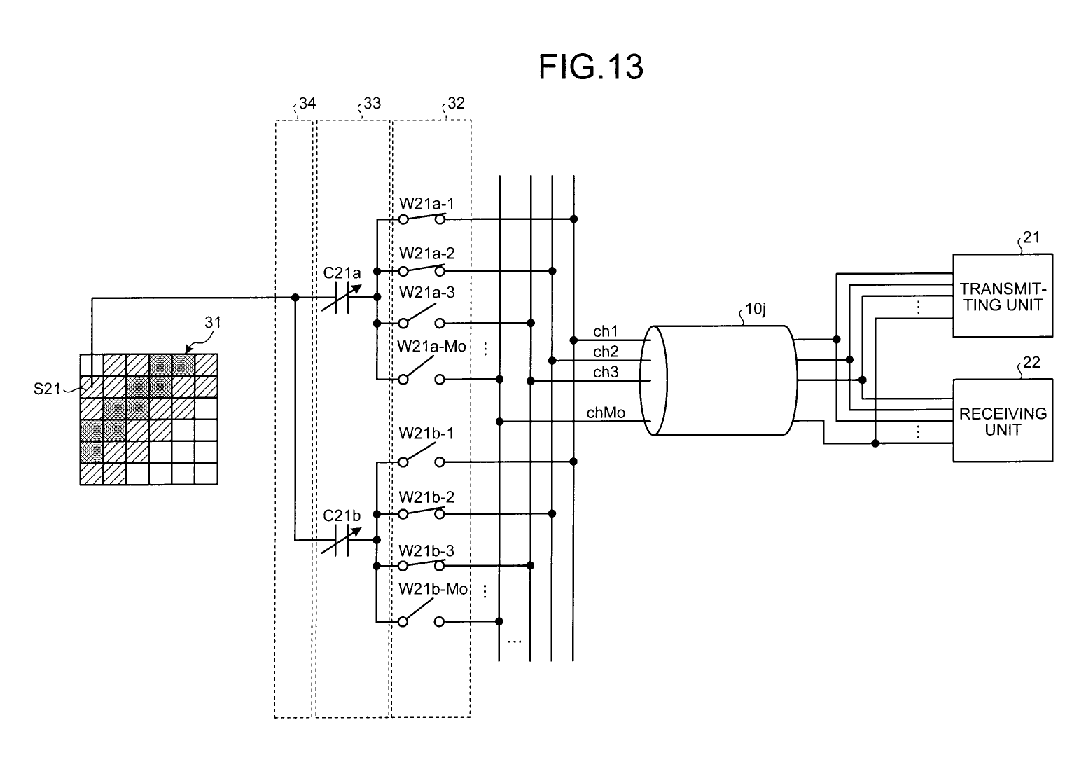

Next, a specific example of the signal selecting unit 32 that selects a driving signal to be supplied to each of transducer elements of which the quantity is equal to Mx (where Mx is 64×64=4096), out of driving signals that are in as many channels as Mo (where Mo=128) and are output from the transmitting and receiving unit 2 included in the apparatus main body 1 will be explained, with reference to FIGS. 13 to 17.

As explained above, to drive each of the transducer elements that form the transducer element group 31 and of which the quantity is equal to Mx, the signal selecting unit 32 selects adjacent driving signals that are in two channels and each of which has a time delay closest to the time delay (i.e., the ideal time delay) required to drive the transducer element, out of the driving signals that are in as many channels as Mo and are supplied from the transmitting and receiving unit 2 included in the apparatus main body 1 while the time delays thereof are quantized by Δτ. Further, as explained above, in the case where the maximum time delay that is required to perform an ultrasonic transmitting/receiving process with respect to a direction at a maximum deflection angle θP is expressed as τmax, it is possible to determine the number of channels Mo in this situation by using the expression Mo=τmax/Δτ.

Next, an example will be explained in which an ultrasonic transmitting/receiving process is performed with respect to the direction at the maximum deflection angle θP (where θP=27 degrees) by using the transducer element group 31 that has an aperture D (D=11.5 millimeters) and in which transducer elements of which the quantity is equal to Mx (where Mx=64×64=4096) are arranged in a two-dimensional array with an array interval d (d=0.18 millimeters). In the present example, when the acoustic velocity in a living body is assumed to be Co (where Co=1530 meters/second), it is possible to calculate the maximum time delay of τmax that is required to perform the ultrasonic transmitting/receiving process described above by using an expression τmax≈D tan(θP)/Co=5.1 microseconds. Further, the center frequency of the ultrasonic pulse is expressed as fo (where fo=5 Megahertz), whereas the quantized time delay of Δτ is assumed to satisfy Δτ=To/4=1/4fo (where To: the cycle of the ultrasonic pulse). In this situation, the number of channels Mo of the first driving signals satisfies Mo=102. Accordingly, driving signals that are in at least 102 channels and have the time delays quantized by Δτ are required.

In the following sections, an example will be explained in which driving signals in 128 channels that are supplied from the transmitting and receiving unit 2 included in the apparatus main body 1 are supplied to the transducer elements of which the quantity is 4096. In this situation, it would not be realistic, in terms of the scale of the circuit, to use the method shown in FIG. 6 by which 128 variable capacity devices are provided for each of the transducer elements, because the ultrasonic probe 3 would need to have 524,288 variable capacity devices built therein. Accordingly, it is appropriate to use a method by which, as shown in FIG. 13, adjacent driving signals that are in two channels and each of which has a time delay closest to the time delay (i.e., the ideal time delay) required to drive a corresponding one of the transducer elements are selected out of the driving signals in the 128 channels, so that a variable capacity device that applies a weight to the adjacent driving signals is provided in correspondence with the transducer element.

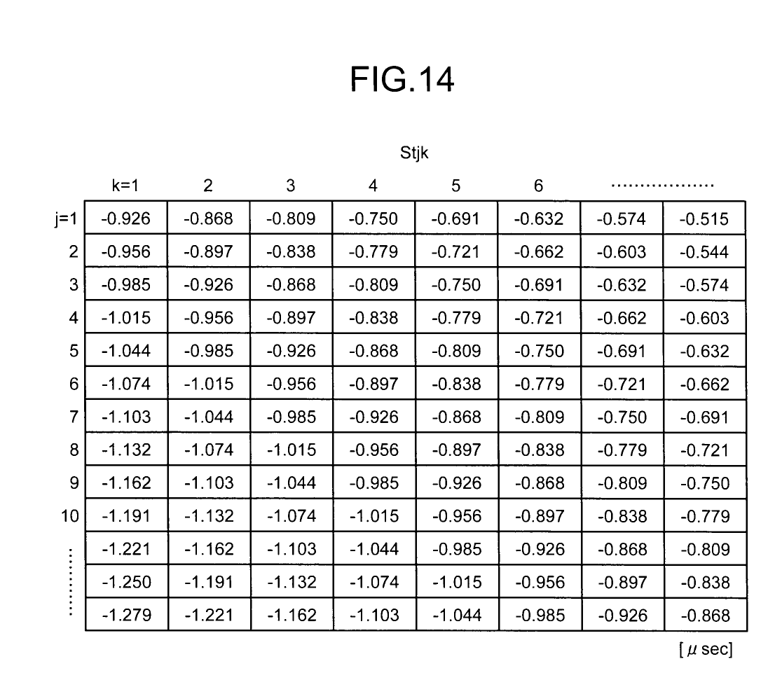

Next, a specific circuit configuration of the signal selecting unit 32 that selects, for each of the transducer elements, adjacent driving signals in two channels, out of the driving signals in the 128 channels will be explained, with reference to FIGS. 14 to 17.

Shown in FIG. 14 are ideal time delays that can be set for a synthesized driving signal Stjk(j,k) to drive a transducer element Sjk (where j=1 to 64 and k=1 to 64) when an ultrasonic pulse is transmitted at the maximum deflection angle θP (where θP=27 degrees). For example, as shown in FIG. 14, by setting a time delay of −0.926 microseconds for the synthesized driving signal to be supplied to the transducer element S11 and setting time delays of −0.868 microseconds and −0.956 microseconds for the synthesized driving signals to be supplied to the transducer element S12 and the transducer element S21, respectively, the signal selecting unit 32 is able to emit an ultrasonic pulse having an excellent directivity property into the transmission/reception direction θP.

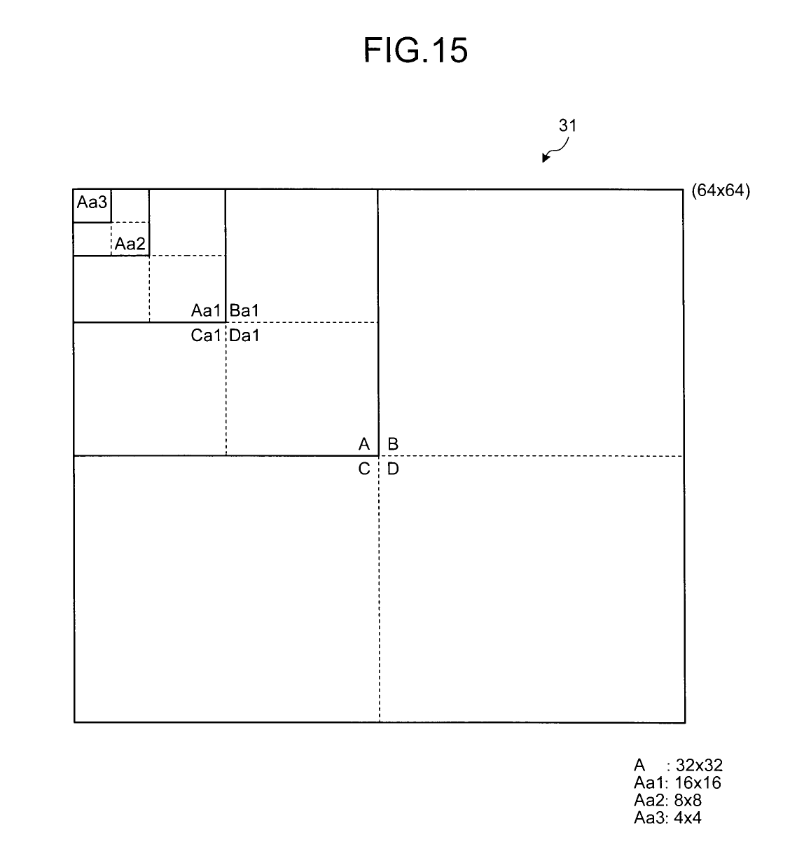

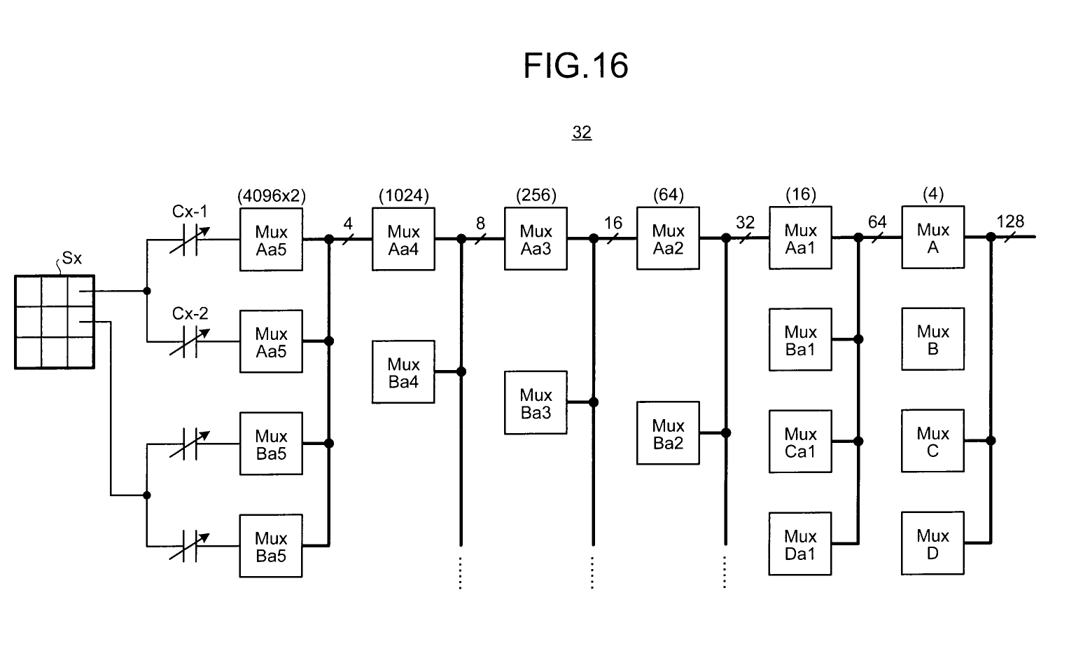

FIG. 15 is a drawing for explaining a method for identifying transducer elements to be driven by sequentially dividing the transducer element group 31 into sections, when the synthesized driving signals for the 4096 transducer elements are generated based on the driving signals in the 128 channels that are supplied from the transmitting and receiving unit 2 included in the apparatus main body 1. An exemplary circuit of the signal selecting unit 32 shown in FIG. 16 is configured in association with the dividing of the transducer element group 31 as shown in FIG. 15.

More specifically, in FIG. 15, the transducer element group 31 formed by 4096(=64×64) transducer elements is divided into four transducer element sub-groups A to D each of which is made up of 32×32 transducer elements. Further, the transducer element sub-group A is divided into transducer element sub-groups Aa1 to Da1 each of which is made up of 16×16 transducer elements. Further, the transducer element sub-group Aa1 is divided into transducer element sub-groups Aa2 to Da2 each of which is made up of 8×8 transducer elements. Further, the transducer element sub-group Aa2 is divided into transducer element sub-groups Aa3 to Da3 each of which is made up of 4×4 transducer elements. Further, the transducer element sub-group Aa3 is divided into transducer element sub-groups Aa4 to Da4 (not shown) each of which is made up of 2×2 transducer elements. Furthermore, each of the transducer element sub-groups B to D is also divided into sections by performing the same procedure.

As shown in FIG. 16, for example, the signal selecting unit 32 is configured with a plurality of multiplexers (MUXs) that separate driving signals and synthesize reception signals. Multiplexers Mux A to Mux D correspond to the transducer element sub-groups A to D, respectively. Further, multiplexers Mux Aa1 to Mux Da1 correspond to the transducer element sub-groups Aa1 to Da1, respectively; multiplexers Mux Aa2 to Mux Da2 correspond to the transducer element sub-groups Aa2 to Da2, respectively; multiplexers Mux Aa3 to Mux Da3 correspond to the transducer element sub-groups Aa3 to Da3, respectively; and multiplexers Mux Aa4 to Mux Da4 correspond to the transducer element sub-groups Aa4 to Da4, respectively.

For example, in the case where the driving signals in the 128 channels of which the time delays are quantized by Δτ are supplied from the transmitting and receiving unit 2 included in the apparatus main body 1, the multiplexer Mux A selects driving signals in 64 channels that are required to drive the 1024 transducer elements forming the transducer element sub-group A, out of the supplied driving signals. Further, the multiplexer Mux Aa1 selects driving signals in 32 channels that are required to drive the 256 transducer elements forming the transducer element sub-group Aa1, out of the driving signals in the 64 channels that have been selected by the multiplexer Mux A.

Further, the multiplexer Mux Aa2 selects driving signals in 16 channels that are required to drive the 64 transducer elements forming the transducer element sub-group Aa2, out of the driving signals in the 32 channels that have been selected by the multiplexer Mux Aa1. Further, the multiplexer Mux Aa3 selects driving signals in 8 channels that are required to drive the 16 transducer elements forming the transducer element sub-group Aa3, out of the driving signals in the 16 channels that have been selected by the multiplexer Mux Aa2. Further, the multiplexer Mux Aa4 selects driving signals in 4 channels that are required to drive the 4 transducer elements forming the transducer element sub-group Aa4, out of the driving signals in the 8 channels that have been selected by the multiplexer Mux Aa3.

Subsequently, the multiplexer Mux Aa5 selects adjacent driving signals in 2 channels each having a time delay closest to the ideal time delay, out of the driving signals in the 4 channels that have been selected by the multiplexer Mux Aa4. After predetermined weights are applied by variable capacity devices Cx1 and Cx2 included in the weighting unit 33 to the adjacent driving signals that have been selected by the multiplexer Mux Aa5, an additive synthesizing process is performed thereon, so that the synthesized driving signal having the ideal time delay is generated.

In other words, the multiplexers Mux A, Mux Aa1, Mux Aa2, Mux Aa3, Mux Aa4, and Mux Aa5 select the adjacent driving signals in two channels each having a time delay closest to the time delay (i.e., the ideal time delay) required to drive the transducer element Sx, out of the driving signals in the 128 channels of which the time delays are quantized by Δτ. Further, the synthesized driving signal for each of the other transducer elements forming the transducer element group 31 is generated by performing the same procedure.

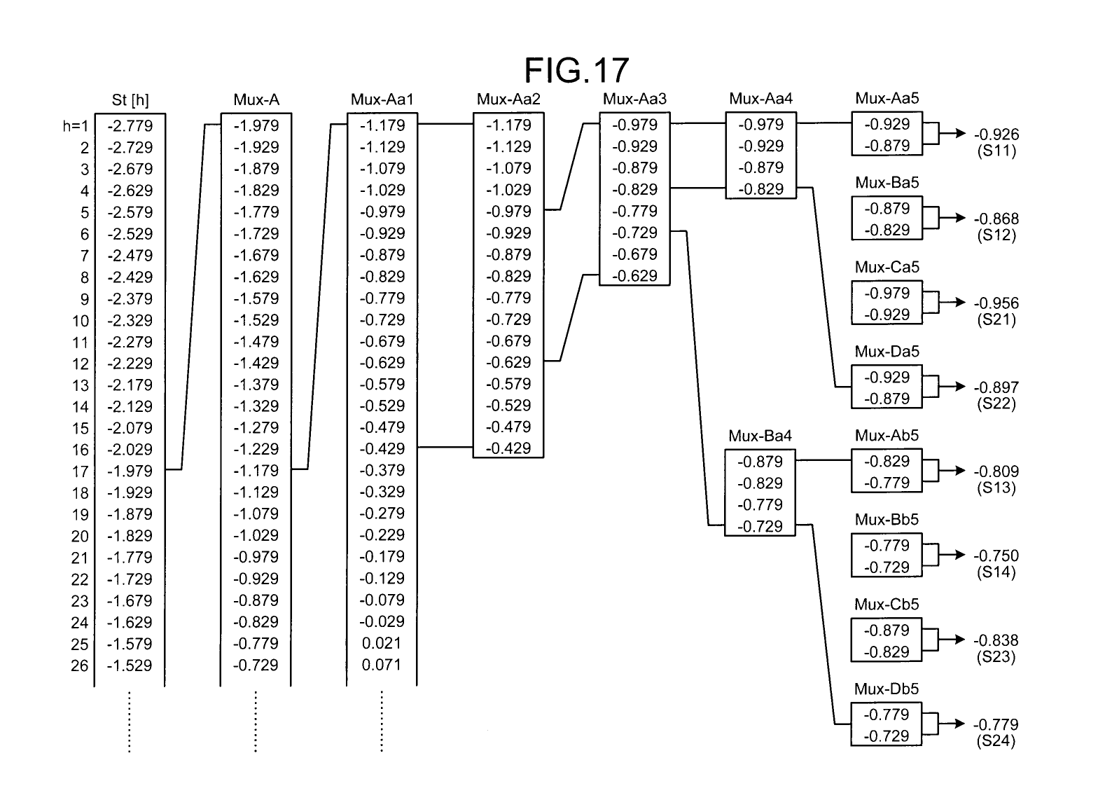

By indicating the respective time delays thereof, shown in FIG. 17 are driving signals St(h) (where h=1 to 128) in 128 channels of which the time delays are quantized by Δτ (where Δτ=To/4=0.05 microseconds) and that are input to the signal selecting unit 32 included in the ultrasonic probe 3 from the transmitting and receiving unit 2 included in the apparatus main body 1, as well as driving signals that are selected by the multiplexers Mux A, Mux Aa1, Mux Aa2, Mux Aa3, Mux Aa4, and Mux Aa5 described above when the synthesized driving signals having the ideal time delays shown in FIG. 14 are generated based on the driving signals St(h).

For example, to generate a synthesized driving signal that has the ideal time delay of −0.926 microseconds and that is used for driving the transducer element S11, the multiplexer Mux A selects driving signals in 64 channels having time delays ranging from −1.979 microseconds to 1.171 microseconds, respectively, out of the driving signals St(h) (where h=1 to 128), whereas the multiplexer Mux Aa1 selects driving signals in 32 channels having time delays ranging from −1.179 microseconds to 0.371 microseconds, respectively, out of the driving signals in the 64 channels. Further, the multiplexer Mux Aa2 selects driving signals in 16 channels having time delays ranging from −1.179 microseconds to −0.429 microseconds, respectively, out of the driving signals in the 32 channels, whereas the multiplexer Mux Aa3 selects driving signals in 8 channels having time delays ranging from −0.979 microseconds to −0.629 microseconds, respectively, out of the driving signals in the 16 channels.

Further, the multiplexer Mux Aa4 selects driving signals in 4 channels having time delays ranging from −0.979 microseconds to −0.829 microseconds, respectively, out of the driving signals in the 8 channels, whereas the multiplexer Mux Aa5 selects adjacent driving signals in 2 channels having time delays of −0.929 microseconds and −0.879 microseconds, respectively, that are closest to the ideal time delay of −0.926 microseconds, out of the driving signals in the 4 channels. Subsequently, the weighting unit 33 and the synthesizing and separating unit 34 generate the synthesized driving signal having the ideal time delay of −0.926 microseconds by weighting and adding together the adjacent driving signals in the two channels that have been selected by the multiplexer Mux Aa5.

In the description above with reference to FIGS. 13 to 17, the example has been explained in which the driving signal to be supplied to each of the transducer elements of which the quantity is equal to Mx is selected out of the driving signals that are in as many channels as Mo and are supplied from the apparatus main body 1. Further, by performing the same procedure as described above, it is possible to perform the selecting/synthesizing processes to organize the reception signals (i.e., the first reception signals) that are in as many channels as Mx and are obtained from the transducer elements into the reception signals (i.e., the second reception signals) in as many channels as Mo.

According to the exemplary embodiments described above, when an ultrasonic transmitting/receiving process is performed on a subject while using the plurality of transducer elements that are arranged in a two-dimensional array within the ultrasonic probe, it is possible to control, with a high level of precision, the time delays of the driving signals to be supplied to the transducer elements or of the reception signals obtained from the transducer elements, while keeping small the number of channels of the signal lines connecting the apparatus main body and the ultrasonic probe together. As a result, it is possible to obtain image data and biological information that have a good quality, by using the ultrasonic probe having excellent operability.

In particular, it is possible to easily generate the synthesized driving signal having the ideal time delay by selecting the adjacent driving signals that are in two channels and each of which has a time delay closest to the ideal time delay required to drive a corresponding one of the transducer elements, out of the driving signals that are in as many channels as Mo, have time delays quantized by Δτ, and are supplied from the transmitting and receiving unit included in the apparatus main body and further weighting and adding together the adjacent driving signals. Further, with the arrangement in which the driving signals of which the time delays are quantized by a quarter of the cycle of the ultrasonic pulse are supplied to the ultrasonic probe 3, the ultrasonic probe 3 is able to generate, without fail, the synthesized driving signal having the ideal time delay for each of the transducer elements. If the time delays were in a rougher unit than a quarter of the cycle of the ultrasonic pulse, it would be impossible to generate a synthesized driving signal having an arbitrary phase, and a side lobe (i.e., an unnecessary response) would occur as a result. In other words, because the transmitting unit 21 according to the exemplary embodiments of the present invention supplies, to the ultrasonic probe 3, the driving signals of which the time delays are quantized by a value smaller than a quarter of the cycle of the ultrasonic pulse, it is possible to inhibit occurrence of side lobes.

Further, according to an aspect of the present embodiments, while keeping the signal selecting unit, the weighting unit, and the synthesizing and separating unit in the same state as when a transmitting process is performed, it is possible to organize the reception signals (i.e., the first reception signals) that are in as many channels as Mx and have been obtained from the transducer element group into the reception signals (i.e., the second reception signals) that are in as many channels as Mo and of which the time delays are quantized by Δτ. As a result, it is possible to accurately perform the phase-matching and adding process in the phase-matching and adding unit that is included in the transmitting and receiving unit where the time delays quantized by Δτ are supplied.

Accordingly, it is possible to achieve an excellent transmission directivity property of the ultrasonic pulse and an excellent reception directivity property of the ultrasonic reflected wave and to generate image data having excellent spatial resolution performance and excellent contrast resolution performance.

Further, according to the exemplary embodiments described above, it is possible to select the adjacent driving signals and to weight and add together the adjacent driving signals by using a relatively small number of circuit elements. Thus, it is possible to reduce the electric power consumption and the heat generation in the ultrasonic probe. As a result, it is possible to efficiently drive the transducer elements and to generate image data having an excellent S/N ratio.

Further, because the weighting process is performed on the adjacent driving signals by using the variable capacity devices, it is possible to significantly reduce the electric power consumption during the weighting process, compared to an example in which resistors are used in the weighting process. Further, because the variable capacity devices (MEMS) are provided by introducing the micromachine technique, it is possible to allow the transducer elements to have a larger number of channels without the need to increase the size or the scale of the ultrasonic probe. In particular, because it is possible to make a stray capacitance smaller by introducing the MEMS, it is possible to realize extremely small variable capacity devices that have a withstand voltage of 100 volts or higher and have a wide capacitance variable range as well as a high response speed.

Further, according to the exemplary embodiments described above, it is possible to arbitrarily and continuously update the time delays of the reception signals obtained from the transducer elements, by using the applied voltages supplied to the positions between the electrodes of the variable capacity devices. As a result, it is possible to easily realize a so-called reception dynamic focus by which the focus point of an ultrasonic reflected wave is moved to a deeper part in conjunction with reception timing of the ultrasonic reflected wave.

Some exemplary embodiments have been explained above. But, it is possible to implement the embodiments while applying modifications thereto. For instance, in the description of the exemplary embodiments above, the example is explained in which the B-mode image data and the color Doppler image data that are two-dimensional on an arbitrary sliced cross-sectional plane are generated by using the ultrasonic probe 3 in which the plurality of transducer elements are arranged in a two-dimensional array; however, according to another aspect of the present embodiments, the image data generated by using the ultrasonic probe 3 in which a plurality of transducer elements are arranged in a two-dimensional array may be other types of image data such as tissue Doppler image data that is obtained by changing a filter constant of the MTI filter 425. Furthermore, it is possible to apply the present embodiments to other examples in which three-dimensional image data, MPR image data, MIP image data or the like is generated, based on volume data obtained by performing a three-dimensional scanning process while using the ultrasonic probe 3.

Further, in the description of the exemplary embodiment above, the ultrasonic probe 3 for a sector scanning purpose is explained; however, the ultrasonic probe 3 may be an ultrasonic probe that is capable of performing a linear scanning process, a convex scanning process, or the like. In particular, in the case where an ultrasonic probe that is capable of performing a linear scanning process or a convex scanning process is used, it is possible to move the aperture used during an ultrasonic transmitting/receiving process toward a desired direction (i.e., a direction perpendicular to the ultrasonic transmission/reception direction), by using the signal selecting unit 32 that is provided for the purpose of selecting the adjacent driving signals and the like. More specifically, according to an aspect of the present embodiment, within the ultrasonic probe that is capable of performing a linear scanning process or a convex scanning process, it is possible to easily switch, by using the signal selecting unit 32, simultaneously-driven channels into a sequential operation.

Furthermore, in the description of the exemplary embodiments above, the example has been explained in which the transmitting and receiving unit 2 supplies the driving signals that are in as many channels as Mo and of which the time delays are quantized by Δτ to the ultrasonic probe 3, whereas the signal selecting unit 32 organizes the first receptions signals that are in as many channels as Mx and have been obtained from the transducer element group 31 into the second reception signals that are in as many channels as Mo and of which the time delays are quantized by Δτ. According to aspects of the present embodiments, however, it is not necessarily requisite to precisely quantize the time delays by Δτ.

In addition, in the description of the exemplary embodiments above, the example has been explained in which each of the synthesized driving signals having the ideal time delays is generated based on the adjacent driving signals in two channels that have been selected by the signal selecting unit 32. According to another aspect of the present embodiments, however, it is possible to generate a synthesized driving signal by using adjacent driving signals that are in three or more channels.

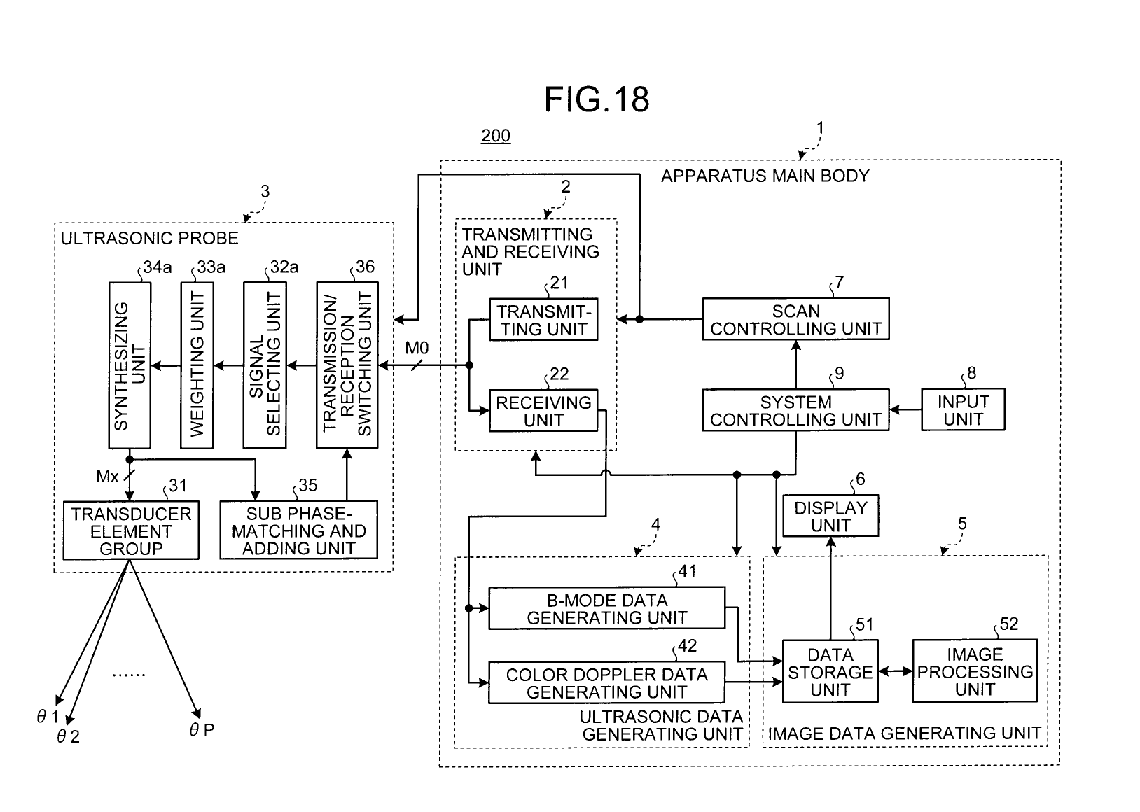

Further, in the description of the exemplary embodiments above, the example has been explained in which the adjacent driving signals are selected during the transmitting process and the reception signals are selected/synthesized during the receiving process, by using the signal selecting unit 32, the weighting unit 33, and the synthesizing and separating unit 34. The present embodiment, however, is not limited to this example. Alternatively, another arrangement is acceptable in which, as shown in FIG. 18, the ultrasonic probe 3 includes: a sub phase-matching and adding unit 35 that is dedicated for receiving processes and that organizes the first reception signals in as many channels as Mx into the second reception signals in as many channels as Mo; and a transmission/reception switching unit 36 that switches between transmissions and receptions.

Furthermore, in the description of the exemplary embodiments above, the ultrasonic diagnostic apparatus that performs the ultrasonic transmitting/receiving process on a subject by using the ultrasonic probe 3 in which the plurality of transducer elements are arranged in a two-dimensional array has been explained; however, the present invention is applicable to an ultrasonic diagnostic apparatus in which a plurality of transducer elements are arranged in a one-dimensional array.

In addition, in the description of the exemplary embodiments above, the example has been explained in which the variable capacity devices are provided on the silicon substrate by introducing the micromachine technique; however, the present embodiment is also applicable to a configuration where the transducer elements forming the transducer element group 31 and/or the circuit device included in the signal selecting unit 32 are also provided on the same silicon substrate by using a micromachine technique. When this configuration is used, it is even easier to allow the transducer elements to have a larger number of channels.

While certain embodiments have been described, these embodiments have been presented by way of example only, and are not intended to limit the scope of the invention. Indeed, the novel apparatuses described herein may be embodied in a variety of other forms; furthermore, various omissions, substitutions and changes in form of the apparatuses described herein may be made without departing from the spirit of the invention. The accompanying claims and their equivalents are intended to cover such forms or modifications as would fall within the scope and spirit of the invention.

What is claimed is:

1. An ultrasonic diagnostic apparatus comprising:
an ultrasonic probe including a plurality of transducer elements that are arranged in an array and each of which is configured so as to transmit an ultrasonic pulse into an ultrasonic transmission/reception direction that is set for an examined subject and to convert an ultrasonic reflected wave arriving from the ultrasonic transmission/reception direction into a reception signal;
a transmitting unit that supplies driving signals that are in a plurality of channels and have discrete time delays to the ultrasonic probe;
a receiving unit that performs a phase-matching and adding process on second reception signals that are in a plurality of channels, have discrete time delays, and have been generated based on first reception signals obtained from the transducer elements; and
an image data generating unit that generates image data based on ultrasonic data obtained by processing reception signals resulting from the phase-matching and adding process, wherein
the ultrasonic probe includes:
a signal selecting unit that selects, as adjacent driving signals, driving signals that are in at least two channels and each of which has a time delay close to an ideal time delay required to drive each of the transducer elements, out of the driving signals that are in the plurality of channels and are supplied from the transmitting unit;
a weighting unit that applies a predetermined weight to each of the selected adjacent driving signals; and
a synthesizing and separating unit that generates a synthesized driving signal having the ideal time delay by synthesizing the adjacent driving signals to which the predetermined weights have been applied and drives each of the transducer elements.

2. The ultrasonic diagnostic apparatus according to claim 1, wherein
the synthesizing and separating unit separates each of the first reception signals that are respectively obtained from the transducer elements into reception signals in at least two channels,
the weighting unit applies a predetermined weight to each of the reception signals resulting from the separation, and
the signal selecting unit generates the second reception signals by selecting and synthesizing the reception signals that have respectively been obtained from the transducer elements and to which the predetermined weights have been applied.

3. The ultrasonic diagnostic apparatus according to claim 2, wherein the weighting unit applies the predetermined weight to each of the driving signals or to each of the reception signals by employing a variable capacity device of which a capacitance is controllable by using a voltage applied thereto.

4. The ultrasonic diagnostic apparatus according to claim 3, wherein the weighting unit applies the predetermined weight to each of the driving signals or to each of the reception signals by employing the variable capacity device that is provided on a silicon substrate by introducing a micromachine technique.

5. The ultrasonic diagnostic apparatus according to claim 4, wherein one or both of the transducer elements and a circuit device included in the signal selecting unit are provided, by introducing the micromachine technique, on the silicon substrate on which the variable capacity device is provided.

6. The ultrasonic diagnostic apparatus according to claim 1, wherein the transmitting unit supplies, to the ultrasonic probe, the driving signals that are in the plurality of channels and of which the time delays are quantized by a predetermined value.

7. The ultrasonic diagnostic apparatus according to claim 6, wherein the transmitting unit supplies, to the ultrasonic probe, the driving signals having the time delays that are quantized by a quarter of a cycle of the ultrasonic pulse transmitted from each of the transducer elements.

8. The ultrasonic diagnostic apparatus according to claim 1, wherein the transmitting unit supplies, to the ultrasonic probe and via a multi-core cable, the driving signals that are in the channels of which a quantity is smaller than a quantity of the transducer elements.

9. The ultrasonic diagnostic apparatus according to claim 1, wherein the signal selecting unit supplies, to the receiving unit included in a main body of the ultrasonic diagnostic apparatus and via a multi-core cable, the second reception signals that are in the channels of which a quantity is smaller than a quantity of the transducer elements.

10. The ultrasonic diagnostic apparatus according to claim 1, wherein the weighting unit sets a time delay for a reception dynamic focus by continuously updating a capacitance of the variable capacity device in correspondence with times at each of which the ultrasonic reflected wave is received.

11. The ultrasonic diagnostic apparatus according to claim 1, wherein the signal selecting unit moves a position of an aperture that is used during an ultrasonic transmitting/receiving process toward a desired direction, by selecting, out of the transducer elements, one or more transducer elements to which the synthesized driving signal is supplied or one or more transducer elements from which the second reception signals are obtained.

12. An ultrasonic probe comprising:
a transducer element group including a plurality of transducer elements that are arranged in an array and each of which is configured so as to transmit an ultrasonic pulse into an ultrasonic transmission/reception direction that is set for an examined subject and to convert an ultrasonic reflected wave arriving from the ultrasonic transmission/reception direction into a reception signal;
a signal selecting unit that selects, as adjacent driving signals, driving signals that are in at least two channels and each of which has a time delay close to an ideal time delay required to drive each of the transducer elements, out of driving signals that are in a plurality of channels, have discrete time delays, and are supplied from a main body of an ultrasonic diagnostic apparatus via a multi-core cable;
a weighting unit that applies a predetermined weight to each of the selected adjacent driving signals; and
a synthesizing and separating unit that generates a synthesized driving signal having the ideal time delay by synthesizing the adjacent driving signals to which the predetermined weights have been applied and drives each of the transducer elements.

13. The ultrasonic probe according to claim 12, wherein
the synthesizing and separating unit separates each of first reception signals that are respectively obtained from the transducer elements into reception signals in at least two channels,
the weighting unit applies a predetermined weight to each of the reception signals resulting from the separation, and
the signal selecting unit generates second reception signals that are in a plurality of channels and have discrete time delays, by selecting and synthesizing the reception signals that have respectively been obtained from the transducer elements and to which the predetermined weights have been applied.

14. The ultrasonic probe according to claim 13, wherein the weighting unit applies the predetermined weight to each of the driving signals or to each of the reception signals by employing a variable capacity device of which a capacitance is controllable by using a voltage applied thereto.

15. The ultrasonic probe according to claim 14, wherein the weighting unit applies the predetermined weight to each of the driving signals or to each of the reception signals by employing the variable capacity device that is provided on a silicon substrate by introducing a micromachine technique.

16. The ultrasonic probe according to claim 15, wherein one or both of the transducer elements and a circuit device included in the signal selecting unit are provided, by introducing the micromachine technique, on the silicon substrate on which the variable capacity device is provided.

17. The ultrasonic probe according to claim 12, wherein the signal selecting unit supplies, to a receiving unit provided in the main body of the ultrasonic diagnostic apparatus and via a multi-core cable, second reception signals that are in channels of which a quantity is smaller than a quantity of the transducer elements.

18. The ultrasonic probe according to claim 12, wherein the weighting unit sets a time delay for a reception dynamic focus by continuously updating a capacitance of the variable capacity device in correspondence with times at each of which the ultrasonic reflected wave is received.

19. The ultrasonic probe according to claim 12, wherein the signal selecting unit moves a position of an aperture that is used during an ultrasonic transmitting/receiving process toward a desired direction, by selecting, out of the transducer elements, one or more transducer elements to which the synthesized driving signal is supplied or one or more transducer elements from which the second reception signals are obtained.
